(19) United States
(12) Patent Application Publication
(10) Pub. No.: US 2024/0225600 A1
(43) Pub. Date: Jul. 11, 2024

(54) SYSTEM FOR OPTIMIZING AN IMAGING APERTURE

(71) Applicant: OneProjects Design and Innovation Ltd., Dublin (IE)

(72) Inventors: Christoph Hennersperger, München (DE); Cédric Assambo, Dublin (IE)

(21) Appl. No.: 18/404,231

(22) Filed: Jan. 4, 2024

Related U.S. Application Data
(60) Provisional application No. 63/437,428, filed on Jan. 6, 2023.

(51) Int. Cl. A61B 8/00; A61B 8/12

(52) U.S. Cl. CPC A61B 8/4488; A61B 8/12; A61B 8/4494; A61B 8/54

(57) ABSTRACT

The invention relates to methods, systems, and devices providing an imaging array having a row-column addressed configuration and capable of being controlled via bias activation, thereby allowing the selection and optimization of an imaging aperture for ultrafast imaging.

### CROSS-REFERENCE TO RELATED APPLICATIONS

This application claims priority to, and the benefit of, U.S. Provisional Application No. 63/437,428, filed Jan. 6, 2023, the content of which is incorporated by reference herein in its entirety.

### FIELD OF THE INVENTION

The invention generally relates to ultrasound imaging, and, more particularly, to systems and devices providing an imaging array having a row-column addressed configuration and capable of being controlled via bias activation, thereby allowing the selection and optimization of an imaging aperture for ultrafast imaging.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image.

For example, catheter-based endovascular ultrasound imaging technology employed within the vasculature (e.g. intravascular ultrasound (IVUS) or intracardiac echocardiography (ICE)) is commonly performed with two-dimensional (2D)-Ultrasound Imaging. In IVUS/ICE imaging systems, an ultrasonic transducer assembly is attached to a distal end of a catheter. The catheter is carefully maneuvered through a patient's body to an area of interest, such as within a coronary artery (for the case of IVUS), or within the right atrium (for the case of ICE). The transducer assembly transmits ultrasound waves and receives echoes from those waves. The received echoes are then converted to electrical signals and transmitted to processing equipment, in which a resulting ultrasound image of the area of interest may be displayed.

Conventional 2D ultrasound imaging has been widely used because it can dynamically display 2D images of the region of interest in real-time. While 2D Endovascular Ultrasound is the standard of care, it requires the operator to know the anatomy at hand for navigation. This requires a high amount of dexterity in maneuvering the catheter image plane to visualize the target structure for a specific interventional use-case. Thus, both the catheter and the imaging plane must be concurrently maneuvered. 2D imaging is also limited to displaying a slice of the anatomy only.

Further, in typical ultrasound systems configured to visualize inner body regions, dynamic forces are often employed, resulting in a dynamic movement of the body regions over time. These dynamic forces and movements make it difficult to stabilize internal imaging devices and to generate consistent and accurate images if imaging of the structure cannot be enabled in real-time (e.g., >20 Hz). As a result, the captured images often lack the necessary quality required to prescribe appropriate treatment or therapy. Because of the dynamic forces and movements in play, internal real-time imaging is limited to small two-dimensional areas or, as noted below, three-dimensional volumetric regions respectively.

2D array transducers have enabled three-dimensional (3D) ultrasound imaging. 3D ultrasound imaging was developed to address the drawbacks of 2D ultrasound imaging and to help diagnosticians and interventionalists acquire a full understanding of the spatial anatomic relationship. In particular, physicians can view an arbitrary plane of the reconstructed 3D volume, as well as panoramic view of the region of interest. Thus, 3D imaging can yield a superior depiction of target structures as well as evaluation, e.g. volumetric assessment.

However, 3D imaging systems have drawbacks and limitations. For example, 3D imaging systems provide a view of the region of interest that is limited to a pyramidical volume (e.g., a trapezoid fan angle that is either side- or forward-looking from the catheter), which is further limited to a 90-degree by 60-degree sector opening for advanced imaging catheters. As such, while 2D array transducers have enabled 3D ultrasound imaging, difficult engineering tradeoffs still exist between system complexity and achievable image quality. Thus, while 3D ultrasound imaging offers significant promise for a wide range of clinical applications, clinical impact is currently limited, in part because image quality is often inferior to 2D imaging using linear- or phased-array transducers. Imaging fully around a catheter in a 360-degree field of view can overcome the limitations of the 2D and 3D limited field of view as described above, and thus enable clinical users delivering better therapy.

### SUMMARY

The present invention recognizes the drawbacks of current 3D ultrasound systems, namely the technical challenges that limit the implementation of a full 360-degree view ultrasound catheter probe for 3D imaging without compromising image quality. In particular, a major challenge of current systems is the connection between the imaging electronics such as application-specific integrated circuits (ASICs), and a flexible ultrasound transducer matrix array, which leads to wiring congestion for fully wired high-density 2D arrays as the channel number increases. In contrast to rigid arrays, in which the array can be integrated with an underlying imaging or processing ASIC directly, a flexible array does not allow for integration within a rigid ASIC underneath geometrically, and thus signal interconnections are more challenging for such a design.

For example, to achieve a flexible imaging array capable of full circumferential imaging, sampling requires high element counts in both a lateral direction (along a cylinder length of the array) with the center to center distance of two adjacent elements (pitch) to be smaller than ½ wavelength, and also a in elevational direction (around the cylinder circumference of the array) with angular spacing determined by wave propagation. These requirements result in tens of thousands of elements leaving limited space for electronics such that individual connections to each element are not possible. The number of channel connections required quickly becomes impossible for large arrays or for invasive probes where cable and probe sizes must be small. Furthermore, wired transmission of all data is not possible without consolidating multiple signals (digital or analog) into a single composite signal (multiplexing). Additionally, wireless transmission of raw data, for example from an ASIC in a catheter tip, with all elements receiving in parallel, requires several tens of gigabytes per second. Because the data rate requirements are enormous, wireless data transmission is also not possible.

The present invention addresses such drawbacks by providing systems, devices and methods providing for high-quality full 360-degree ultrafast imaging using a novel imaging array architecture that avoids subsampling of the array and extensive processing in hardware. Specifically, devices of the invention include an imaging array and system architecture that achieves ultrafast imaging within the in-plane and out-of-plane aperture, specifically optimizing an imaging aperture based on row-column addressing schemes in combination with bias voltage tuning that provides high-performance, flexible, and fully software-defined imaging. Accordingly, the systems, methods, and devices of the invention provide for flexible imaging arrays capable of transmitting and receiving ultrafast ultrasound signals to achieve full 360-degree view imaging without compromising on image quality.

In specific embodiments, the invention provides systems and devices for optimizing an imaging aperture to provide high resolution ultrafast imaging. For example, the invention provides an imaging array design using row (or column) activation in combination with bias tuning of a row-column array structure, enabling high-quality and flexible ultrasound imaging, particularly for cylindrical array applications.

Particularly, systems, devices, and methods of the invention use a novel system architecture for row-column addressing in combination with bias voltage tuning, i.e. activation. Using row-column addressing to address each individual imaging element of the imaging array limits the number of interconnects by enabling addressing of each element as a combination of a row (address) and column (address). Further, the rows and/or columns of the imaging array can be fully activated or deactivated using bias activation for ultrasound transducer arrays such as bias sensitive Capacitive Micromachines Ultrasound Transducers (CMUTs). For example, a nominal bias voltage can be applied to a row of imaging elements to tune the imaging array to a target center frequency. Thus imaging will be activated on this row. Likewise, deactivating bias (e.g. applying 0V or a voltage level where the sensitivity of the element is minimal) deactivates an imaging row. This allows for fully enabling or disabling specific rows and/or columns according to flexible transmit/receive schemes.

Using the principle described above, the use of row-column addressing of imaging elements in the array in combination with bias activation achieves a desired angular aperture for ultrafast imaging. Specifically, applying varying transmit/receive/biasing patterns subsequentially during imaging allows for targeted ultrafast imaging. Thus, the invention provides for defining and optimizing an angular aperture for ultrafast imaging that is not limited to a 90 by 60 degrees sector opening for a pyramidical volume view. As provided by the invention, image aperture optimization applies to any convex array surfaces with a certain radius. For example, this concept may be applied to the special case of a convex surface fully around the imaging probe, i.e. a cylindrical surface. Thus, in some embodiments, the angular imaging aperture may be understood to mean the angular direction along a curvature.

Aspects of the present invention include systems for optimizing an imaging aperture comprising a controller operably associated with an imaging device. In particular, the imaging device includes a transducer having an array of individual imaging elements arranged as a plurality of rows longitudinally along the transducer and as a plurality of columns laterally along the cylindrical transducer, such that the signal connectivity of an individual imaging element is defined by a row address position and a column address position of the array. The imaging device further includes a plurality of first electrodes each in connection with a row of individual imaging elements, and a plurality of second electrodes arranged at a non-zero angle relative to the plurality of first electrodes each in connection with a column of individual imaging elements.

The controller is coupled to non-transitory, computer-readable memory containing instructions executable by the processor to cause the controller to selectively apply a bias voltage level to one or more of the plurality of first and/or second electrodes such that the bias voltage level defines a voltage for the row or column connected to the electrode to activate or deactivate imaging by the individual imaging elements in the row or column to define an angular imaging aperture.

In some embodiments of the systems, the angular imaging aperture is defined by the number of rows (Nr) and/or the number of columns (Nc) to which the bias voltage is applied such that a number of interconnections between imaging elements is Nr+Nc.

In some embodiments, the systems further include a plurality of interface modules such that each interface module is operably connected to both the controller and to one electrode in connection with a row or column. Further, a bias voltage level for each row and/or each column is individually enabled, disabled, and set using the interface module for the row or column. Particularly, to activate a desired row of imaging elements, the controller selects a transmit address corresponding to the desired row and sends a signal with a desired bias voltage level to the interface for the desired row. Further, in some embodiments, to activate a desired column of imaging elements, the controller selects a transmit address corresponding to the desired column and sends a signal to the interface with a desired voltage level for the desired column. In other embodiments, the interface further comprises one or more ORing diodes in series with a digital to analog converter (DAC) output to protect each imaging element from simultaneous activation of bias voltage at its row address position and column address position. In some embodiments, the ORing circuits utilize diodes and/or transistors.

In some embodiments, the controller is configured to activate single or multiple rows at once using the same or different bias voltage levels to achieve an intended angular imaging aperture. Additionally and/or alternatively, the controller is configured to activate single or multiple columns at once using the same or different bias voltage levels to achieve an intended angular imaging aperture.

Further, in some embodiments of systems of the invention, the controller includes a computer program comprising an algorithm for evaluating, calculating, and optimizing the angular imaging aperture. Thus, the algorithm may be used such that the controller is configured to activate single or multiple rows at once using the same or different bias voltage levels to achieve the optimized angular imaging aperture. Likewise, the controller is configured to activate single or multiple columns at once using the same or different bias voltage levels to achieve the optimized angular imaging aperture. In some embodiments, the controller may also be capable of controlling transmit and receive wave patterns for flexible image formation, or these capabilities may be integrated otherwise.

In some embodiments, the controller simultaneously activates multiple individual imaging elements based on the row address position and column address position of the respective individual imaging elements. Further, the simultaneously activated individual imaging elements have the same or different bias voltage levels according to embodiments of the systems. In some embodiments, the bias voltage selectively applied to one or more of the plurality of first and/or second electrodes enables or disables a transmit and/or receive function to define a transmit-receive event wherein each transmit-receive event comprises an activation and/or a tuning scheme. Further, in embodiments, the controller is configured to control the activation and/or tuning scheme individually for each transmit-receive event such that multiple transmit-receive events may have the same or alternating activation and/or tuning scheme.

Also according to some embodiments of the system, the controller includes a protection circuit operably connected in series with each row and each column of individual imaging elements such that multiple bias voltage levels cannot be simultaneously applied to a given electrode. In other embodiments, the controller also includes an integrated circuit housed within an enclosure together with the imaging elements or positioned upon a substrate together with the imaging elements.

In particular examples of the systems, the controller is housed separately from the imaging elements and is operably coupled to the imaging elements via circuitry. For example, the circuitry includes at least one of one or more cable assemblies, one or more printed circuits, and/or one or more flexible printed circuits in some embodiments of the systems.

Further, in some embodiments of the system, the controller includes an integrated circuit for bias voltage generation and control, wherein the integrated circuit is housed in an enclosure with the imaging elements or positioned upon a substrate with the imaging elements, and an analog front-end circuit, housed separately from the imaging elements, and comprising one or more signal generators, and/or one or more signal transmitters, and one or more switching circuits. In examples, the integrated circuit is housed together with the analog front-end circuit at a catheter tip adjacent to the imaging elements. In other embodiments, the integrated circuit is housed together with the analog front end circuit operably coupled to the imaging elements and positioned directly adjacent to the imaging elements. Further, in various representations of the systems, the switching circuits, the signal transmitters, and the switching circuits are housed in a remote enclosure connected to the imaging elements via circuitry comprising one or more cable assemblies, one or more printed circuits, and/or one or more flexible printed circuits.

Notably, in some embodiments of the systems of the invention, the bias voltage level is adjustable to tune a frequency of the imaging elements. In examples, the bias voltage level selectively applied to the plurality of first and second electrodes is the same or different for each electrode to allow for tuning the imaging frequency of the imaging elements higher and/or lower to achieve a desired frequency. In other examples of the systems, a positive bias voltage and/or a negative bias voltage level selectively applied to the plurality of first and/or second electrodes tunes an imaging operating mode.

In particular representations of the systems, applying a bias voltage of 0V or a voltage level where a sensitivity of the imaging elements is minimal deactivates imaging in the row or column. For example, in some embodiments, one or more columns and/or one or more rows are deactivated.

In some embodiments, the angular imaging aperture is one or more rows or columns defined based on a beam opening sensitivity of an individual imaging element of the array. Further, the angular imaging aperture is defined as one row or column up to about 10 rows or columns in particular embodiments.

Further, in some embodiments of the systems, the transducer array is a micro-electromechanical systems (MEMS)-based capacitive micromachined ultrasonic transducer (CMUT) configured as a two-dimensional (2D) array structure in particular embodiments of the systems. Further, in examples, the 2D array structure is a flexible structure. The transducer may also include an electrostrictive material configured as a two-dimensional (2D) array structure in some embodiments of the systems.

In specific embodiments of the systems of the invention, the plurality of imaging elements are acoustic sensors selectively activated by the controller based on the row address and/or the column address of the acoustic sensor to receive a plurality of incident acoustic wave signals as wave data. For example, the wave data comprises at least one of plane wave data and/or diverging wave data associated with one or more plane wave transmit-receive cycles carried out by the imaging elements, in particular embodiments of the systems. Further, the wave data may be full circumferential, three-dimensional (3D) image data.

In some embodiments, the transducer is cylindrically shaped. This provides for an embodiment of the systems in which the array of individual imaging elements is arranged as a plurality of rows longitudinally along the transducer and as a plurality of columns circumferentially around the transducer. Thus, the array comprises a number of rows (Nr), a number of individual imaging elements per row (Ne), a row spacing, and a number of columns (Nc), wherein the total number of imaging elements in the array is (Ne·Nr) and the individual imaging elements are connected through a number of connections represented by Nr+Nc, in some embodiments of the systems. For example, in some embodiments, the row spacing is from about 0.1 degree to about 5 degrees in angular direction.

In example representations of the systems of the invention, the bias voltage is applied to a first electrode to activate a transmit and/or a receive function on the individual imaging elements connected to the second electrode such that the individual imaging elements transmit and/or receive ultrasound signals in the form of ultrafast wave data. Advantageously, the transducer array includes a number of individual imaging elements per row (Ne) in an array design allowing ultrafast plane wave and/or diverging wave imaging, such that the plane wave and/or diverging wave imaging mode comprises capturing plane wave reflected signal data at a rate of at least 10 kHz.

In other embodiments of systems of the invention, the plurality of second electrodes is arranged orthogonally relative to the plurality of first electrodes.

In some embodiments, one or more bias voltage selection circuits are connected to the controller using one or more multipoint communication interfaces.

In some embodiments, the control of the transmit function and/or the receive function uses one or more interfaces that are common to or separate from the interfaces used for bias voltage selection.

In further aspects, the invention provides methods for optimizing an imaging aperture, for example, for high resolution ultrafast imaging. The method includes providing a controller operably associated with an imaging device. In particular, the imaging device includes a transducer having an array of individual imaging elements arranged as a plurality of rows longitudinally along the transducer and as a plurality of columns laterally along the cylindrical transducer, such that the signal connectivity of an individual imaging element is defined by a row address position and a column address position of the array.

The imaging device further includes a plurality of first electrodes each in connection with a row of individual imaging elements, and a plurality of second electrodes arranged at a non-zero angle relative to the plurality of first electrodes each in connection with a column of individual imaging elements.

The controller is coupled to non-transitory, computer-readable memory containing instructions executable by the processor to cause the controller to selectively apply a bias voltage level to one or more of the plurality of first and/or second electrodes. The methods further include applying a bias voltage level to one or more selected electrodes such that the bias voltage level defines a voltage for the row or column connected to the electrode to activate or deactivate imaging by the individual imaging elements in the row or column to define an angular imaging aperture.

In some embodiments of the methods, the angular imaging aperture is defined by the number of rows (Nr) and/or the number of columns (Nc) to which the bias voltage is applied such that a number of interconnections between imaging elements is Nr+Nc.

In some embodiments, the method further includes a plurality of interface modules such that each interface module is operably connected to both the controller and to one electrode in connection with a row or column. Further, a bias voltage level for each row and/or each column is individually enabled, disabled, and set using the interface module for the row or column, in some embodiments of the methods. Particularly, to activate a desired row of imaging elements, the controller selects a transmit address corresponding to the desired row and sends a signal with a desired bias voltage level to the interface for the desired row. Further, in some embodiments, to activate a desired column of imaging elements, the controller selects a transmit address corresponding to the desired column and sends a signal to the interface with a desired voltage level for the desired column. In still other embodiments, the interface further comprises one or more ORing diodes in series with a digital to analog converter (DAC) output to protect each imaging element from simultaneous activation of bias voltage at its row address position and column address position. In some embodiments, the ORing circuits utilize diodes and/or transistors.

Importantly, in some embodiments, the controller is configured to activate single or multiple rows at once using the same or different bias voltage levels to achieve an intended angular imaging aperture. Additionally and/or alternatively, the controller is configured to activate single or multiple columns at once using the same or different bias voltage levels to achieve an intended angular imaging aperture.

In some embodiments of the methods, the controller further includes a computer program comprising an algorithm for evaluating, calculating, and optimizing the angular imaging aperture. In specific embodiments, the controller is configured to activate single or multiple rows at once using the same or different bias voltage levels to achieve the optimized angular imaging aperture. Additionally and/or alternatively, the controller is configured to activate single or multiple columns at once using the same or different bias voltage levels to achieve the optimized angular imaging aperture. In some embodiments, the controller may also be capable of controlling transmit and receive wave patterns for flexible image formation, or these capabilities may be integrated otherwise.

In some embodiments, the controller simultaneously activates multiple individual imaging elements based on the row address position and column address position of the respective individual imaging elements. Further, the simultaneously activated individual imaging elements have the same or different bias voltage levels according to embodiments of the methods. In embodiments, the bias voltage selectively applied to one or more of the plurality of first and/or second electrodes enables or disables a transmit and/or receive function to define a transmit-receive event wherein each transmit-receive event comprises an activation and/or a tuning scheme. Further, in some embodiments, the controller is configured to control the activation and/or tuning scheme individually for each transmit-receive event such that multiple transmit-receive events may have the same or alternating activation and/or tuning scheme.

According to some embodiments of the methods, the controller includes a protection circuit operably connected in series with each row and each column of individual imaging elements such that multiple bias voltage levels cannot be simultaneously applied to a given electrode. In other embodiments, the controller also includes an integrated circuit housed within an enclosure together with the imaging elements or positioned upon a substrate together with the imaging elements.

In particular examples of the methods, the controller is housed separately from the imaging elements and is operably coupled to the imaging elements via circuitry. For example, the circuitry includes at least one of one or more cable assemblies, one or more printed circuits, and/or one or more flexible printed circuits in some embodiments of the methods.

Further, in some embodiments, the controller includes an integrated circuit for bias voltage generation and control, wherein the integrated circuit is housed in an enclosure with the imaging elements or positioned upon a substrate with the imaging elements, and an analog front-end circuit, housed separately from the imaging elements, and comprising one or more signal generators, and/or one or more signal transmitters, and one or more switching circuits. In examples, the integrated circuit is housed together with the analog front-end circuit at a catheter tip adjacent to the imaging elements. In other examples, the integrated circuit is housed together with the analog front end circuit operably coupled to the imaging elements and positioned directly adjacent to the imaging elements. Further, in various representations of the methods, the switching circuits, the signal transmitters, and the switching circuits are housed in a remote enclosure connected to the imaging elements via circuitry comprising one or more cable assemblies, one or more printed circuits, and/or one or more flexible printed circuits.

Notably, in some embodiments of the methods of the invention, the bias voltage level is adjustable to tune a frequency of the imaging elements. In examples, the bias voltage level selectively applied to the plurality of first and second electrodes is the same or different for each electrode to allow for tuning the imaging frequency of the imaging elements higher and/or lower to achieve a desired frequency. In other examples, a positive bias voltage and/or a negative bias voltage level selectively applied to the plurality of first and/or second electrodes tunes an imaging operating mode.

In particular representations of the methods, applying a bias voltage of 0V or a voltage level where a sensitivity of the imaging elements is minimal deactivates imaging in the row or column. For example, in some embodiments, one or more columns and/or one or more rows are deactivated.

In some embodiments of the methods, the angular imaging aperture is one or more rows or columns defined based on a beam opening sensitivity of an individual imaging element of the array. Further, the angular imaging aperture is defined as one row or column up to about 10 rows or columns in particular embodiments.

In some embodiments, the transducer array is a micro-electromechanical systems (MEMS)-based capacitive micromachined ultrasonic transducer (CMUT) configured as a two-dimensional (2D) array structure in particular embodiments of the methods. Further, in examples, the 2D array structure is a flexible structure. The transducer may also include an electrostrictive material configured as a two-dimensional (2D) array structure in some embodiments of the methods.

In specific embodiments of the methods, the plurality of imaging elements are acoustic sensors selectively activated by the controller based on the row address and/or the column address of the acoustic sensor to receive a plurality of incident acoustic wave signals as wave data. The wave data comprises at least one of plane wave data and/or diverging wave data associated with one or more plane wave transmit-receive cycles carried out by the imaging elements, in particular embodiments of the methods. Further, the wave data may be full circumferential, three-dimensional (3D) image data.

In some embodiments of the methods, the transducer is cylindrically shaped. This provides for an embodiment of the methods in which the array of individual imaging elements is arranged as a plurality of rows longitudinally along the transducer and as a plurality of columns circumferentially around the transducer. Further, the array comprises a number of rows (Nr), a number of individual imaging elements per row (Ne), a row spacing, and a number of columns (Nc), wherein the total number of imaging elements in the array is (Ne·Nr) and the individual imaging elements are connected through a number of connections represented by Nr+Nc, in some embodiments of the methods. For example, in some embodiments, the row spacing is from about 0.1 degree to about 5 degrees in angular direction.

In example representations of the methods of the invention, the bias voltage is applied to a first electrode to activate a transmit and/or a receive function on the individual imaging elements connected to the second electrode such that the individual imaging elements transmit and/or receive ultrasound signals in the form of ultrafast wave data. Advantageously, in some embodiments, the transducer array includes a number of individual imaging elements per row (Ne) in an array design allowing ultrafast plane wave and/or diverging wave imaging, such that the plane wave and/or diverging wave imaging mode comprises capturing plane wave reflected signal data at a rate of at least 10 kHz.

In other embodiments of methods of the invention, the plurality of second electrodes is arranged orthogonally relative to the plurality of first electrodes.

In some embodiments, one or more bias voltage selection circuits are connected to the controller using one or more multipoint communication interfaces.

In some embodiments, the control of the transmit function and/or the receive function uses one or more interfaces that are common to or separate from the interfaces used for bias voltage selection.

### BRIEF DESCRIPTION OF THE DRAWINGS

FIGS. 1A and 1B are diagrammatic illustrations of an exemplary ultrasound system for providing visualization and characterization of vasculature within a patient with which devices of the invention may be used.

FIG. 2 is a perspective view of an imaging catheter with which devices of the invention may be coupled.

FIG. 3 is a diagrammatic illustration of the prior art ASIC designs for matrix array probes.

FIG. 4 illustrates a diagram of the physical sensitivity of imaging elements radially around a cylindrical array catheter in some embodiments of the invention.

FIG. 5 illustrates the active radial aperture for radial rows around a cylindrical imaging catheter in an embodiment of the invention.

FIGS. 6A and 6B illustrate a side view and cross section in an embodiment of a cylindrical imaging array of the invention.

FIG. 7 illustrates a flexible imaging array and system architecture that provides row-column addressing in which front electrodes address rings or columns, and back electrodes address rows.

FIG. 8 illustrates an embodiment of a cylindrical imaging array of the invention.

FIG. 9 illustrates classic row-column addressing.

FIG. 10 illustrates an embodiment of bias row activation in devices of the present invention.

FIG. 11 illustrates an embodiment of the system architecture of the invention in which the transmit function may be activated by the bottom electrodes and the receive function may be activated on the top electrodes.

FIG. 12 illustrates a non-limiting example of row activation in an embodiment of a flexible imaging array of the invention.

FIG. 13 illustrates a non-limiting example of column/ring activation in an embodiment of a flexible imaging array of the invention.

FIG. 14 illustrates activation of multiple rows of an embodiment of an imaging array of the invention.

FIG. 15 illustrates frequency tuning of multiple active rows in an embodiment of an imaging array of the invention.

FIG. 16A illustrates a diagram of one embodiment of the interface for a row of elements in an array.

FIG. 16B illustrates an enlarged view of the circuit diagram of FIG. 16A.

FIG. 17A illustrates a diagram of one embodiment of imaging array element interfacing for a specific row and for a specific column, for example row i and column j.

FIG. 17B illustrates an enlarged view of the circuit diagram of FIG. 17A.

FIG. 18A illustrates an embodiment of the invention in which each row of the imaging array is connected to an individual array element interface as is each column of the array such that individual imaging elements may be addressed by a row and column (row, column) address.

FIG. 18B illustrates an enlarged view of the circuit diagram of FIG. 18A.

FIG. 19A illustrates an embodiment of the interfacing circuit with the bias voltage selection showing the interfacing circuit for the imaging element at row i, and column j, i.e. the imaging element at address (i, j).

FIG. 19B illustrates an enlarged view of the circuit diagram of FIG. 19A.

FIG. 20 illustrates an embodiment of an imaging device of the invention in which the controller is in active communication with a computing system comprising an algorithm for evaluating, calculating, and optimizing the angular imaging aperture.

FIG. 21 diagrams another embodiment of systems of the invention to produce an ultrasound image using ultrafast imaging techniques.

FIG. 22 illustrates a cylindrical array transducer with rows of elements radially around the transducer that may be used for imaging a side of the transducer.

FIG. 23 illustrates the activation of a single element.

FIG. 24 is a block diagram of an embodiment of methods of the invention for imaging.

FIG. 25 is a block diagram of an embodiment of methods of the invention for optimizing an imaging aperture.

### DETAILED DESCRIPTION

The present invention recognizes the drawbacks of current 3D ultrasound systems, including the technical challenges limiting the implementation of a full 360-degree view catheter for ultrafast imaging without compromising on image quality. The invention provides systems, devices, and methods for ultrafast ultrasound imaging using uniquely configured imaging array transducers and system architectures. The invention provides systems and devices for optimizing an imaging aperture for full 360-degree ultrafast imaging.

Specifically, the invention provides bias selection and tuning for row activation in combination with row-column addressing to enable flexible imaging protocols specifically for ultrafast imaging modes (planewave and diverging wave). Previous attempts using row-column addressing to define an imaging aperture have focused mainly on focused wave imaging in planar array configurations (classic 2D matrix array setups). Thus, the invention provides for high-performance, high-quality, flexible, and fully software-defined imaging. Imaging devices and systems of the invention are capable of ultrafast imaging that avoids subsampling of the array and extensive processing in hardware and enables flexible imaging protocols and approaches for ultrafast image reconstruction. Systems and devices of the invention are capable of optimizing the imaging aperture, including an angular imaging aperture, providing flexible imaging arrays capable of ultrafast full 360-degree imaging.

By way of overview, and as is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). In an ultrasound exam, the imaging device, (i.e. the transducer, probe, or transducer probe) is placed directly on the skin or inside a body opening (e.g. endovascular ultrasound, intravascular ultrasound, intracardiac echocardiography). The final quality of the image obtained through ultrasound scanning is limited to the technical specifications of the equipment, the propagation of ultrasonic waves through the tissue analyzed, and the method used to reconstruct the images.

### Ultrasound Imaging System

Systems, devices, and methods of the invention address the challenges of interconnect and how to efficiently and flexibly transmit and receive ultrafast ultrasound signals with flexible arrays. Systems and devices of the invention provide novel imaging arrays and corresponding system architectures to solve these problems and provide for ultrafast imaging within the in-plane and out-of-plane aperture.

As described in more detail below, in some embodiments, the invention provides a system for optimizing an imaging aperture using a novel imaging array transducer with row-column addressing and bias activation which enables a lower interconnect while providing flexibility for imaging with convex aperture selection and optimization. In some embodiments, this combination allows for a flexible ultrafast imaging catheter with a cylindrical imaging array in a small form factor and improved imaging contrast, resolution and sensitivity as compared to other catheter systems using 2D planar arrays.

Systems and devices of the invention may be manufactured and/or assembled using current approaches. Systems and devices of the method may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein in their entirety.

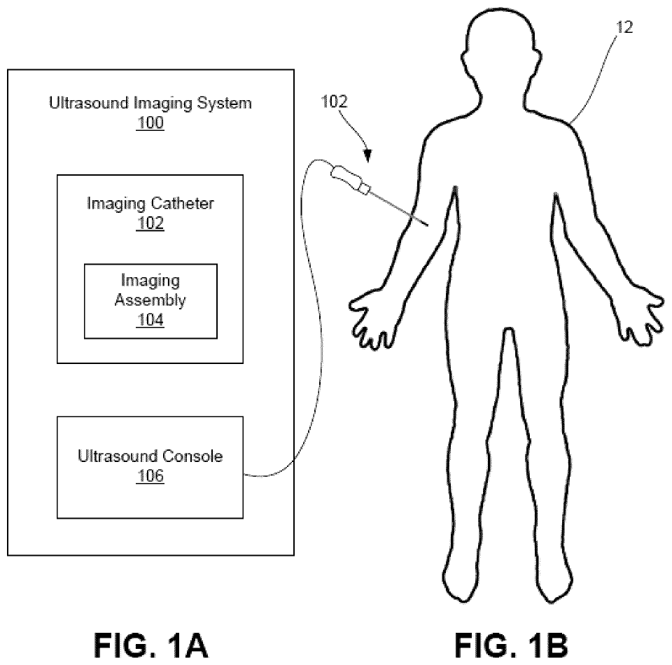

FIGS. 1A and 1B are diagrammatic illustrations of an exemplary ultrasound system 100 for providing visualization and characterization of, for example, vasculature within a patient 12 with which systems and devices of the invention may be used. Generally, the system 100 includes an imaging catheter 102 equipped with an imaging assembly 104 and an ultrasound console 106 to which the imaging catheter 102 is to be connected.

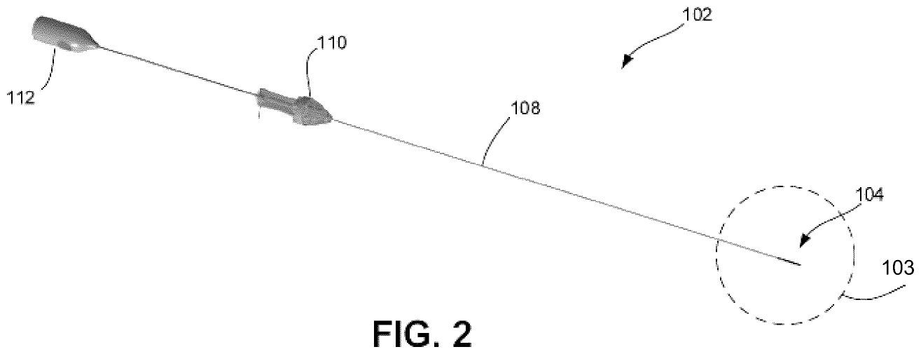

FIG. 2 is a perspective view of an imaging catheter 102 with which the device may be coupled. The catheter 102 may include a catheter body 108, including proximal and distal portions. The imaging assembly 104 may be provided at the distal portion, for example, generally defining a distal end of an imaging catheter 103. A handle 110 may be operably associated with the catheter body 108 and allow for an operator (i.e., surgeon or other medical professional) to manipulate and advance the imaging assembly 104 and the catheter body 108 to a desired target site within the patient's vasculature. The handle 110 may include user-operable inputs for controlling various features and functions of the imaging assembly 104. An interface member 112 may be provided at a distal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

### Ultrafast Ultrasound Imaging

Ultrafast ultrasound imaging techniques, such as planewave or diverging wave imaging, may be required to enable imaging within the constraints of the application, particularly for intravascular and/or intracardiac tissue imaging. Systems, devices, and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position. In contrast to the present invention, other (native 3D) transducer arrays presented in literature for intracardiac imaging do not allow for the generation of full angular aperture imaging, nor do they allow for full 360 coverage around the catheter, both of which are prerequisites for accurate depth and contiguity/permanency monitoring of cardiac treatments such as ablations.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques.

However, ultrafast imaging has been primarily limited to 2D imaging with rigid linear array transducers. While 2D array transducers have enabled 3D ultrasound imaging there exists difficult engineering tradeoffs between system complexity and achievable image quality. This is due, in part, because planewave (or diverging wave) imaging does not use a focal point for the transmission of the ultrasound signal but rather simultaneously excites all elements in the array for the transmission of ultrasound signals at different angles, where focusing on a focal point is performed in software using all received data for one or multiple ultrafast transmit and receive events. As a result, ultrafast imaging requires a full electrical connection from each transducer element to a respective imaging channel, such that all transducer elements are utilized in parallel for both transmit and receive modes.

To achieve a flexible imaging array capable of full circumferential imaging, sampling requires high element counts in both a lateral direction (e.g. along a cylinder length) with pitch d smaller than wavelength, and also in elevational direction (e.g. around a cylinder circumference) with angular spacing a determined by both wavelength as well as angular beam profile. In signal processing, sampling is the reduction of a continuous-time signal to a discrete time signal. Sampling, in this context, means the conversion of a sound wave to a sequence of samples having a value of the signal at a point in time and/or space.

The requirement for high element counts to achieve the requisite sampling results in tens of thousands of elements which leaves limited space for electronics such that connection to each element is not possible. Thus, for an array with N×N channels with N² elements would require N² channel connections, which would quickly become impractical for large arrays or for invasive probes where cable and probe sizes must be small.

To illustrate this point, a center frequency of 8 MHz for a cylindrical array 15 mm in length is considered. For a wavelength of 0.195 mm, the maximum possible pitch d of elements is 0.18 mm (with limited ultrafast capabilities), and an ideal pitch being smaller than 0.0975 mm. This results in 84 lateral imaging elements (154 elements respectively for the ideal case) per row (Nr). For a full circumferential array with elevational spacing of 1 degree, the total elevational imaging elements per column (Nc) is 360. The total number of elements (Nr×Nc) is 30,249. To connect each element requires a total number of connections that is currently not possible and leaves limited space for driving electronics. Thus a wired transmission of all data is not possible without consolidating multiple signals (digital or analog) into a single composite signal (multiplexing) that is then transmitted.

As a result, implementation of fully wired arrays is currently prohibitive, for example, with commercial (nonmicrobeamformer) arrays available with only a limited number of elements. Using smaller arrays to address the challenge of interconnects results in small aperture sizes which limits the achievable spatial resolution and contrast, specifically in miniature applications, resulting in poor or limited image quality. Larger probes with high element density may be used to address image quality and produce high-quality images. However, the larger number of channels leads to significant interconnect and channel count difficulties and are not practical for miniature applications. A few approaches have been attempted to reduce the channel count while having a larger aperture size, such as multiplexing and sparsely distributing the active elements, with limited channels but these methods have demonstrated sidelobe artifacts that degrade image quality.

Certain steps, such as sub-aperture beamforming (micro-beamforming), can be directly integrated with the imaging array to simplify the imaging interconnect. However, while this may work for scanline-based or focused imaging approaches, the performance for ultrafast (planewave/diverging wave) sequences deteriorates significantly with this configuration, as sub-aperture beamforming assumes the transmission of focused waves with a given focal point, for which delays can be applied within a sub-aperture to create constructive wave interference and thus effective sub-aperture beamforming.

Monolithically integrating a full imaging chain with the imaging array in order to process data fully at the array before transferring it in a digital and processed form to a system has also been used. However, due to the processing in hardware, this limits the flexibility and adaptability for using different imaging modes, and also creates significant challenges for large arrays in terms of heat dissipation and thermal management (both of which remain as significant challenges in miniature probes).

Row-column addressing arrays have been proposed to limit the required cabling and enable certain individual elements to be triggered by combining transmission on rows and reception on columns. However, previously proposed imaging designs have suffered from speed and/or image quality issues and have only been successful for rigid (non-flexible) 2D arrays, for example as described in Sobhani, 2022, Ultrafast orthogonal row-column electronic scanning (uFORCES) with bias-switchable top-orthogonal-to-bottom electrode 2D arrays, IEEE Trans Ultrason Ferroelect Freq Cont 69(10):2823-2835.

Further, while planewave and diverging wave imaging eliminate the need to form a scan line, and cover a wider area, the loss of signal-to-noise ratio in the image resolution make it necessary to have higher data processing and filtering to improve these parameters. For example, wireless transmission of raw data from an ASIC in a catheter tip, with all elements receiving in parallel, requires several tens of gigabytes per second transmitted in real-time. Because the data rate requirements are enormous, wireless data transmission is not possible for the required distance between a probe in the body, and an outside device. In the example above, the raw data rate would be 141 Mbyte per firing which results in several tens of gigabytes per second, which is not possible for standard wireless transmission.

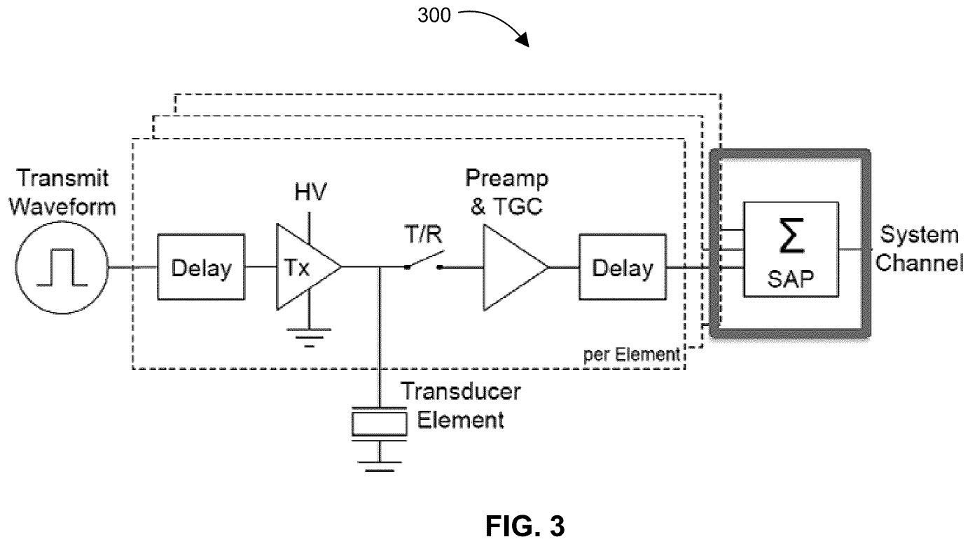

FIG. 3 illustrates a prior art ASIC design 300 for matrix array probes. These designs have smaller groups of elements, such as sub-aperture groups, sub-aperture processors, etc. These subgroups perform local delay and sum (receive beamforming) to limit the required data channels to the receive side. This limits the flexibility for beamforming and can only support pre-designed delay schemes such as scanline, pulse echo, and pulsed doppler.

Thus, in order to achieve high quality image resolution in ultrafast imaging, a major challenge of current application-specific integrated circuits (ASICs) in a flexible matrix array ultrasound system is the wiring congestion necessary for fully wired high-density 2D arrays as the channel number increases.

### Novel Imaging Array

The present invention recognizes that having a good sampling for a flexible, ultrasound imaging array capable of ultrafast imaging requires high imaging element counts. In contrast to rigid arrays where the array can be integrated with an underlying imaging or processing ASIC directly, such as through the same process technology or through flip chip bonding, a flexible array does not allow for integration within a rigid ASIC underneath geometrically, and thus signal interconnect is more challenging for such a design.

Systems, devices, and methods of the invention address the challenges of interconnect and how to efficiently and flexibly transmit and receive ultrafast ultrasound signals with flexible arrays. Systems and devices of the invention provide novel imaging arrays and corresponding system architectures to solve these problems and provide for ultrafast imaging within the in-plane and out-of-plane aperture.

Specifically, systems and devices of the invention include imaging arrays and system architectures capable of row-column addressing and bias aperture selection for ultrafast imaging. As described in more detail below, systems and devices of the invention use bias selection and tuning for row (or column) activation in combination with row-column addressing to define an angular imaging aperture that enables flexible imaging protocols specifically for ultrafast imaging modes (planewave and diverging wave). Prior art attempts at row-column addressing have been successful only for rigid 2D arrays and focused wave imaging.

Systems and devices of the invention provide for accessing specific imaging elements as needed without carrying out fixed operations in subaperture groups. Thus, for arrays of the invention, element groups do not have sub-aperture beamforming, but instead receive all data at once. Importantly, the invention provides for row-column activation for any number of rows and/or columns on the imaging array to define an angular imaging aperture. This allows the activation of single or multiple rows at once depending on the intended and/or evaluated imaging aperture, particularly with circumferential apertures. This is not possible with classical signal multiplexing circuits.

Additionally, different bias voltages may be used to tune the imaging frequency higher or lower, or combine adjacent rows and/or columns with differently tuned frequencies for harmonic tuning.

### Angular Imaging Aperture

Systems and devices of the invention provide for flexible imaging arrays capable of ultrafast imaging. To define and optimize an angular imaging aperture, systems and devices of the invention allow for accessing the imaging elements as needed without carrying out fixed operations in sub-aperture groups. Importantly, using row-column addressing to define (i.e. tune) an imaging aperture of the array, particularly an angular imaging aperture, applies to any convex array surfaces with a certain radius. For example, this concept may be applied to the special case of a convex surface fully around the imaging probe, i.e. a cylindrical surface. Thus, in some embodiments, the angular imaging aperture may be understood to mean the angular direction along a curvature.

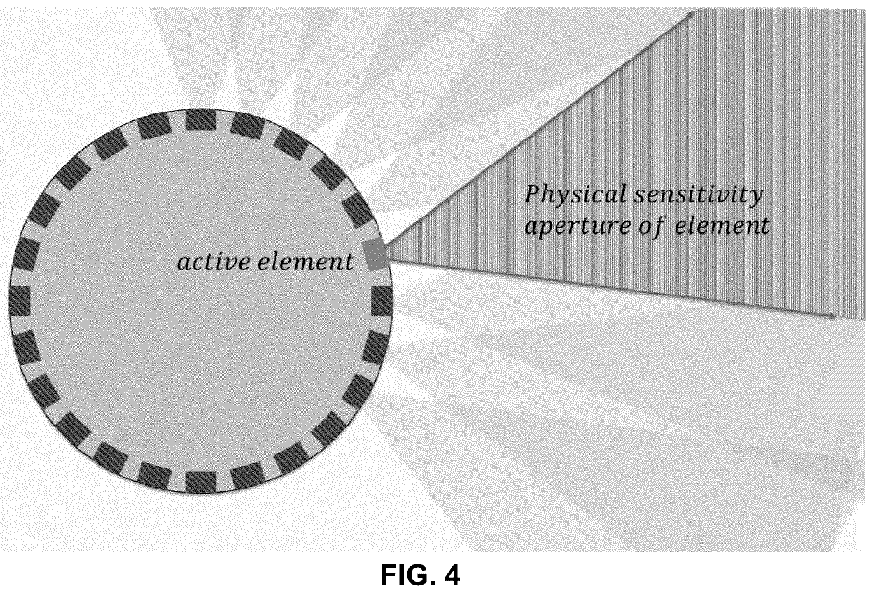

FIG. 4 illustrates a diagram of the physical sensitivity of imaging elements radially around a cylindrical array catheter in some embodiments of the invention. The cylindrical shape (or radius of the cylinder) provides geometrical boundary constraints to which elements can receive data for a point in space. For an active element, the physical sensitivity aperture of the element is illustrated. Sensitivity describes the electro-transducer energy conversion efficiency and is a key indicator of transducer performance. Generally, sensitivity is defined as the ratio of an output quantity to an input quantity.

The aperture is the active area that transmits or receives acoustic wave at certain moment. For a single-element transducer, the aperture size is the transducer element size. For a transducer with an array elements, the aperture is all the elements that are active simultaneously. As is generally understood, ultrasound imaging has a spatially variant resolution that depends on the size of the active aperture of the transducer (including the dimensions of each ultrasound element), the center frequency and bandwidth of the transducer, and the selected transmit pattern. For focused imaging, lateral resolution is best at the focal length distance and widens away from this distance in a nonuniform way because of diffraction effects caused by apertures on the order of a few to tens of wavelengths. For non-focused imaging such as planewave or diverging wave imaging, lateral resolution for a transmit widens away from the transducer surface, as focusing is performed using multiple transmit waves.

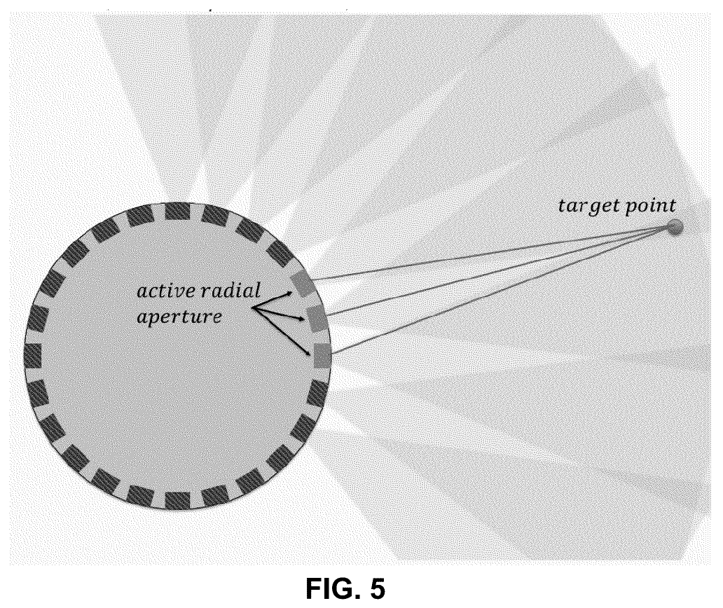

FIG. 5 illustrates the active radial aperture for radial rows around a cylindrical imaging catheter in an embodiment of the invention. The active aperture depends on the angular coverage of each physical element (i.e. the directivity of imaging elements). Each ultrasound transducer has its own specific directivity pattern. The directivity pattern, also known as beam pattern or radiation pattern, is an important far-field characteristic of a transducer. Directivity pattern consists of a main lobe and side lobes. Radiation intensity is dominant mainly in the front region of the transducer source, so the main lobe is directly in front of the ultrasound transmitter, followed by side lobes sidewise with null region in between these lobes. In general, the directivity patterns are the same whether the transducer is used as a transmitter or as a receiver.

The invention provides systems and devices with a flexible imaging array that allow for flexibility in convex aperture selection and optimization. The invention takes advantage of the fact that a convex array, including a cylindrically-shaped array, provides geometrical boundary constraints as to which elements can receive data for a point in space. For example, the angular imaging aperture of a cylinder results in a limited angular coverage of ultrasound waves emitted from and received by a flexible imaging array. Specifically, imaging of a certain side or view from a cylindrical catheter may be sequential and not the full cylinder at once due to this radial aperture. This means that a full sampling of multiple rows around the circumference of the cylindrical array would not yield an improved circumferential resolution.

The invention uses this concept to define an angular aperture of a defined number of rows, for example from one up to a group of rows. In this way, systems and devices of the invention allow for a desired aperture based on the geometry of the ideally emitting ultrasound imaging array. For example, alongside the array, all the elements of the array may be used. Alternatively, out of the plane, the aperture may be, for example, 3-5 rows depending on the pitch of the elements, which enables larger apertures enabling increased circumferential resolution.

This combination of row-column addressing and bias activation and tuning achieves an angular imaging aperture for full 360-degree ultrafast imaging. In specific embodiments, this concept allows for a flexible, ultrafast imaging catheter with a cylindrical imaging array that provides improved imaging contrast, resolution and sensitivity as compared with other catheter systems using planar 2D arrays. Thus, the invention provides for full 360-degree circumferential ultrafast imaging not previously possible.

As is described in detail below, it is noted that systems and devices of the invention are also applicable to planar 2D arrays.

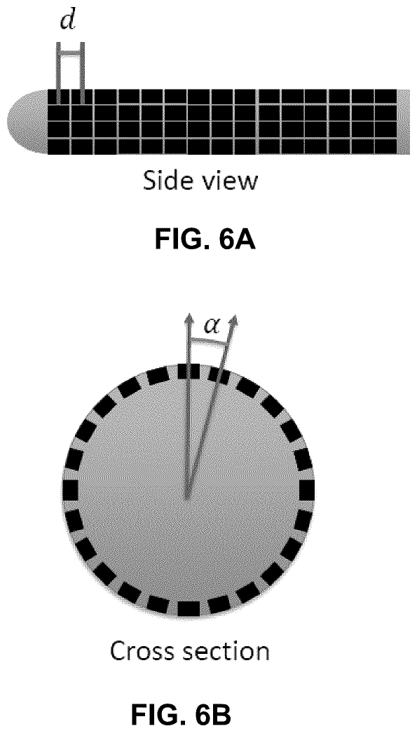

FIGS. 6A and 6B illustrate a side view and cross section in an embodiment of a cylindrical imaging array of the invention. The cylindrical imaging array provides for high element counts in both the lateral direction (e.g. along a cylinder length) with pitch d, and also in elevational direction (e.g. around a cylinder circumference) with angular spacing a. Thus, the flexible imaging array is capable of achieving full circumferential imaging.

The flexible imaging arrays for ultrafast ultrasound imaging may be configured for any shape, for example cylindrical/non-cylindrical, flat and non-flat. As noted above, the flexible arrays of the invention allow for defining and optimizing an angular aperture for any shaped surface but particularly for a concave surface, e.g. a cylindrical array. However, in some embodiments, the system architecture may be used for rigid imaging arrays, for example a rigid convex/cylindrical array capable of defining a specific angular imaging aperture for full 360-degree imaging.

It should be noted that specific descriptions of the present invention focus on using the systems and devices of the invention for ultrasound visualization of intravascular and/or intracardiac tissue, which may be particularly useful for catheter-based interventional procedures for assessing the anatomy as well as functional data in relation to the target volume of interest. However, as is generally understood, the devices, systems, and methods of the present invention may be used for ultrasound visualization of tissue of any kind with respect to any kind of procedure in which imaging analysis is used and/or preferred.

Aspects of the invention provide a system for optimizing an imaging aperture including a controller operably associated with an imaging device. The imaging device includes a transducer comprising an array of individual imaging elements arranged as a plurality of rows longitudinally along the transducer and as a plurality of columns laterally along the transducer. As described in more detail below, the signal connectivity of an individual imaging element is defined by a row address and a column address of the array. The device includes a plurality of first electrodes in connection with a row of individual imaging elements, and a plurality of second electrodes arranged at a non-zero angle relative to the plurality of first electrodes, such that each second electrode is in connection with a column of individual imaging elements. The controller is coupled to non-transitory, computer-readable memory containing instructions executable by the processor to cause the controller to selectively apply a bias voltage level to one or more of the plurality of the first and second electrodes. Thus, the bias voltage level defines a voltage for the row or column connected to the electrode to activate or deactivate imaging by the individual imaging elements in the row or column to define an angular imaging aperture.

In the present invention, the transducer may be any type for transmitting and receiving acoustic waves. For example, the transducer may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices may not require bias for achieving imaging sensitivity.

In some embodiments of the systems of the invention, the transducer may be a micro-electromechanical systems (MEMS)-based capacitive micromachined ultrasonic transducer (CMUT) configured as a two-dimensional (2D) array structure. In non-limiting examples, the 2D array may be a flexible structure. Devices of the invention may include a cylindrical imaging array which consists of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13(10) 3873-3882, incorporated herein by reference.

Additionally, and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers of the invention may be made of electrostrictive materials such as an electrostrictive polymer, or any material that can be activated using bias voltage to achieve imaging sensitivity.

### Row-Column Addressing

The invention uses row-column addressing in combination with bias activation to achieve an angular imaging aperture for ultrafast ultrasound imaging. Specifically, systems of the invention employ row-column addressing to tune an imaging aperture. Instead of connecting each element individually, all elements within one row and column are connected together, allowing addressing of elements in a row-column fashion. For example, front electrodes on the imaging array may address columns (or “rings” on a cylindrical array) and back electrodes on the imaging array address “rows”. However, it should be noted that front and back electrodes of the imaging array may be switched for ring/row addressing respectively.

For example, the plurality of first electrodes may be positioned as back electrodes and the plurality of second electrodes may be positioned as front electrodes. Alternatively, the plurality of first electrodes may be positioned as front electrodes and the plurality of second electrodes may be positioned as back electrodes.

For clarity, “ring” may mean imaging elements arranged circumferentially around a cylindrical array, or alternatively may be referred to as columns on the array. Similarly, “rows” may refer to imaging elements arranged longitudinally along the transducer. Thus individual elements may be referred to as being in rows and/or columns/rings, and each individual element will have a row-column address, for example (i, j).

Row-column addressing enables the system to address each imaging element of the array through a row and column fashion, which reduces the amount of interconnects from Nr×Nc for a fully connected array to Nr+Nc connections for row-column addressing. As an example, an imaging array with 64 rows and 180 columns would require (64×180)=11, 520 connections for a fully connected array. Using row-column addressing of the invention, this array would only require (64+180)=244 connections, thus significantly limiting the number of interconnects required.

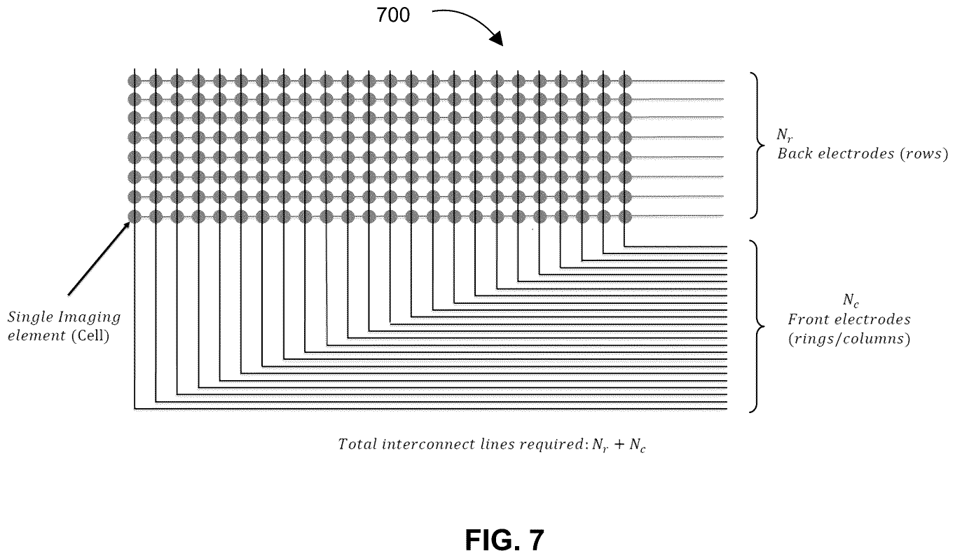

FIG. 7 illustrates an embodiment of an imaging array 700 of the invention. For example, the invention provides a flexible imaging array and system architecture that provides row-column addressing in which front electrodes address rings or columns, and back electrodes address rows. Devices of the invention support ultrafast imaging within the in-plane and out-of-plane apertures.

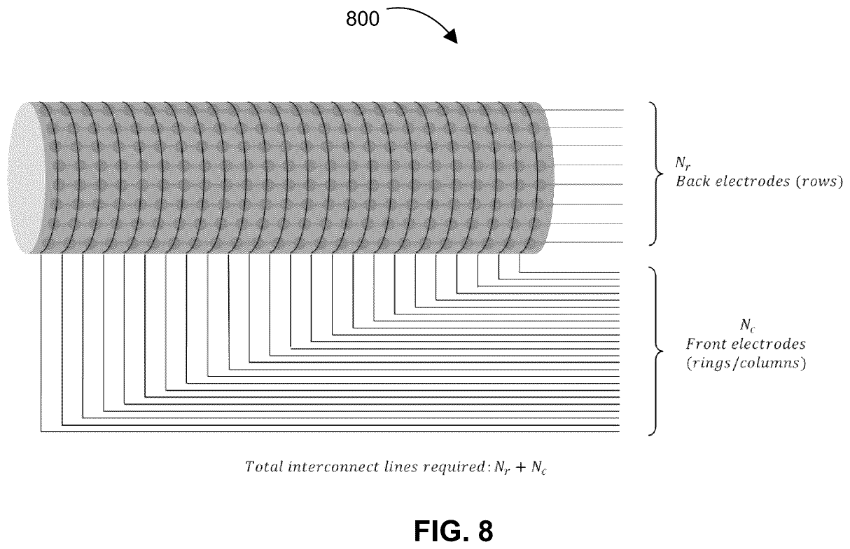

FIG. 8 illustrates an embodiment of a cylindrical imaging array 800 of the invention.

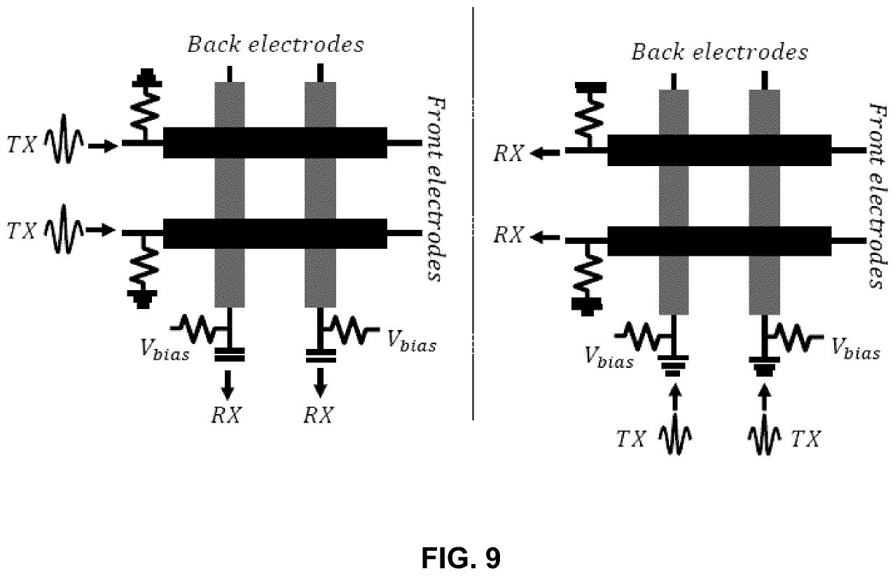

FIG. 9 illustrates classic row-column addressing. In this concept, the transmit function is achieved on one set of electrodes and the receive function is achieved on the orthogonal electrodes, specifically, the transmit function is on a full row, and the receive function is on a column to address a specific imaging element. Until the present invention, classic row-column addressing was only capable for transmit and receive on orthogonally positioned electrodes.

The angular imaging aperture may be defined as one or more rows or one or more columns of the imaging array based on a beam opening sensitivity of an individual imaging element of the array. For example, the angular imaging aperture may be defined as one row or one column up to about 10 rows or 10 columns. The individual beam opening sensitivity may be known such that this value is included in calculations of a desired angular imaging aperture. The individual beam opening sensitivity of an imaging element may be obtained through measurement and/or simulations.

### Bias Activation

The invention takes advantage of the fact that for modern MEMS-based capacitive arrays such as a capacitive micromachined ultrasonic transducer (CMUT), the bias voltage required by CMUT arrays enables not only frequency tuning of the target imaging frequency, but also enables deactivating elements due to the high dampening/sensitivity decrease when bias is deactivated. The invention recognizes that CMUT arrays require bias voltage supplied through one of the electrodes, which may be the back or front electrodes, to operate the CMUT elements efficiently.

The invention provides for using bias voltage to tune the frequency range of the imaging array with the sensitivity for a frequency range being driven by the bias voltage. Bias voltage may be used to deactivate an imaging element entirely. Importantly, using bias voltage to activate imaging elements (i.e. cells) allows for imaging without multiplexing. Thus, the invention provides for using row-column addressing on the back (or front) electrodes to control the bias voltage applied to imaging elements to activate certain element groups dynamically, or to tune their frequency range sensitivity.

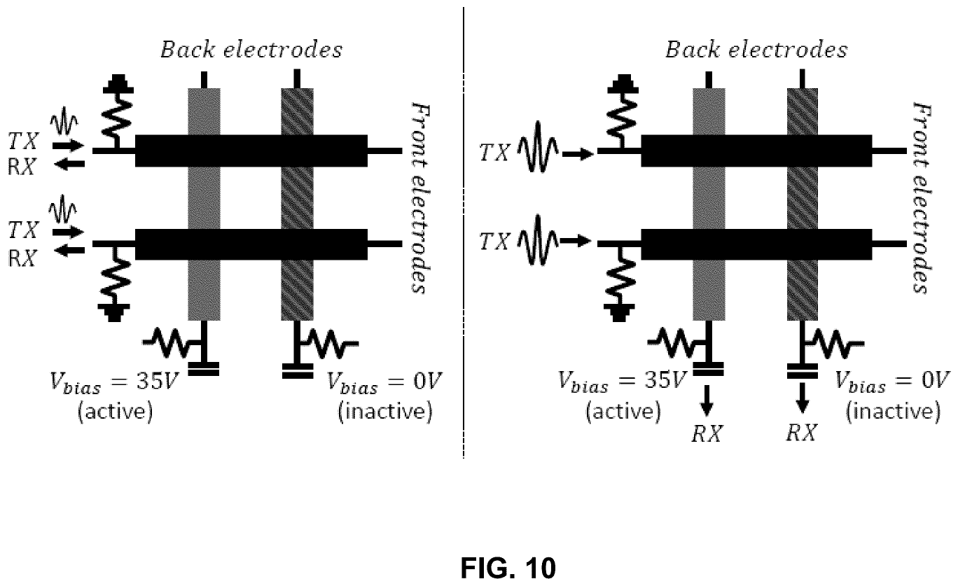

FIG. 10 illustrates an embodiment of bias row activation in devices of the present invention. In contrast to classic row-column addressing, the invention provides for fully activating or deactivating individual rows and columns using bias activation. The invention combines both the concepts of classic row-column addressing and bias row activation to control both the angular imaging aperture and imaging mode, for example, in a CMUT array.

Bias activation means that when a nominal bias voltage is applied to tune the imaging array to a target center frequency imaging will be activated on this row or column. Similarly, applying a bias voltage of 0V or a voltage level where the sensitivity of the imaging element is minimal deactivates the imaging row or column. This allows for the enablement or disablement of full rows or columns on the array and provides for employing a flexible transmit/receive scheme on the activated row(s) and/or column(s).

A bias voltage may be applied to front or back electrodes. As an example, for Nr back electrodes, the electrodes may address rows for transmit and receive. The bias voltage may be applied based on the row address and/or column address of one or more of the individual imaging elements. Additionally, a bias voltage may be applied to activate imaging in one or more rows. Likewise, a bias voltage may be applied to activate imaging in one or more columns.

A bias voltage of 0V may be applied for deactivation of a row, i.e. dampening element sensitivity to −60 dB. For example, applying a bias voltage of 0V or a voltage level where a sensitivity of the imaging elements is minimal may deactivate imaging in the row or column. Further, one or more columns and/or one or more rows may be deactivated.

Additionally, the bias voltage may be adjustable to tune a frequency of the imaging elements. Particularly, the bias voltage selectively applied to the plurality of first and second electrodes may be the same or different for each electrode to allow for tuning the imaging frequency of the imaging elements higher and/or lower to achieve a desired frequency.

For example, a bias voltage of 35V as a nominal voltage may be applied (tuned to 10 MHz imaging) applied to a row. Bias tuning (up or down) may be used to tune a center frequency. Nc front electrodes may address columns/rings for transmit and receive. Thus a transmit pulse may be sent and data may be received for all rings/columns in a selected row. This allows for addressing elements within a row as single elements. As a result, a positive bias voltage and/or a negative bias voltage level selectively applied to the plurality of first and/or second electrodes may tune an imaging operating mode.

Alternatively, the row-column array architecture of the invention may be addressed as a classic row-column array, e.g. transmitting of ultrasound wavs on a selected row and receiving on a selected column. This allows for the use of synthetic-aperture type imaging with a cylindrical array.

Additionally, the bias voltage applied to a first electrode may activate the imaging elements connected to the second electrode for both a transmit function and a receive function.

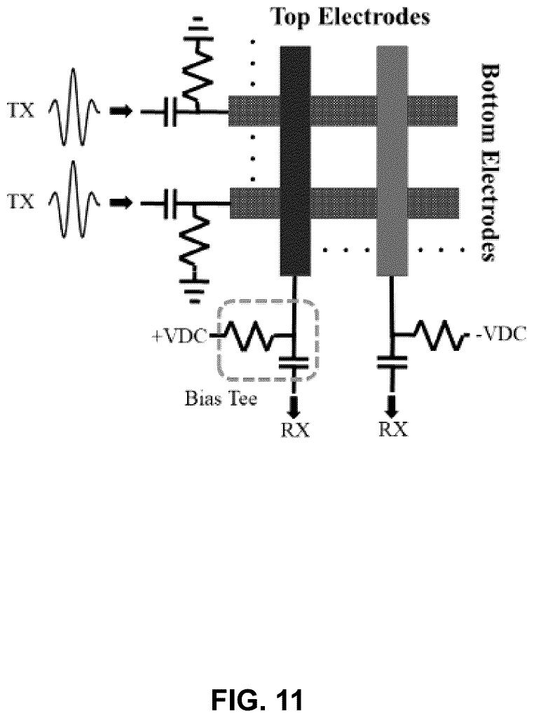

FIG. 11 illustrates an embodiment of the system architecture of the invention in which the transmit function may be activated by the bottom electrodes and the receive function may be activated on the top electrodes. The imaging array system may include a bias tee to supply a DC current or voltage to bias the RF circuit.

Systems of the invention provide devices with a plurality of first electrodes in connection with a row of individual imaging elements, and a plurality of second electrodes arranged at a non-zero angle relative to the plurality of first electrodes. The plurality of second electrodes may be arranged orthogonally relative to the plurality of first electrodes. For example, as illustrated in FIG. 11, transmit and receive functions may be on the same electrode with activation of individual elements through bias (on/off) on the orthogonal electrode. This enables addressing of individual elements by bias voltage in combination with the orthogonal electrode. It should be noted that the transmit and receive functions may be on the same electrode or on orthogonal electrodes.

Importantly, the second set of electrodes may not be orthogonal to the first set of electrodes instead being any non-zero angle in relation to the first set of electrodes. Further, front and/or back electrodes may be switched in terms of interconnect (bias injection versus AC signal for transmit function).

In non-limiting examples, the bias voltage applied to a first electrode may activate (i) a receive function of the individual imaging elements connected to the first electrode and a transmit function of the individual imaging elements connected to the second electrode; or (ii) a transmit function of the individual imaging elements connected to the first electrode and a receive function of the individual imaging elements connected to the second electrode.

Additionally, the bias voltage applied to a second electrode may activate (i) a receive function of the individual imaging elements connected to the second electrode and a transmit function of the individual imaging elements connected to the first electrode; or (ii) a transmit function of the individual imaging elements connected to the second electrode and a receive function of the individual imaging elements connected to the first electrode.

Further, the bias voltage applied to a first electrode may activate a transmit or receive function of the individual imaging elements connected to the first electrode and a transmit or receive function of the individual imaging elements connected to the second electrode.

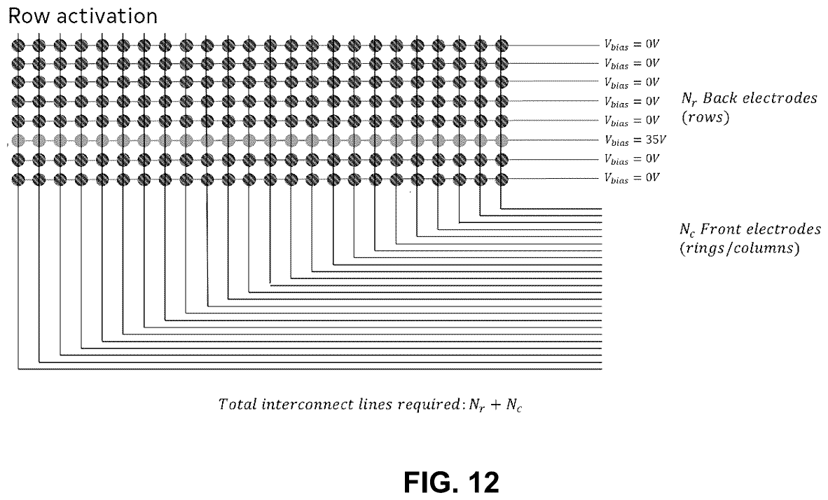

FIG. 12 illustrates a non-limiting example of row activation in an embodiment of a flexible imaging array of the invention. In the example, a bias voltage of 35V may be applied to a selected back electrode to tune the imaging array to a target center frequency such that imaging is activated on this row. The selected surrounding rows may be deactivated by applying a bias voltage of 0V and/or a voltage to these selected rows such that the sensitivity of the imaging elements in these rows is minimal.

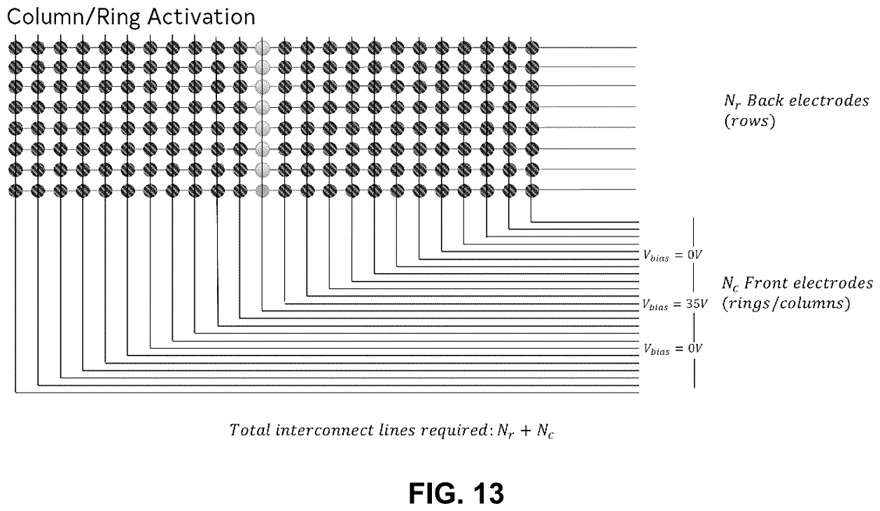

FIG. 13 illustrates a non-limiting example of column/ring activation in an embodiment of a flexible imaging array of the invention. Applying a bias voltage of 0V or a voltage level where a sensitivity of the imaging elements is minimal may deactivate imaging in the row or column. In this example, a bias voltage of 35V may be applied to a select column or ring to activate the column/ring. The selected surrounding columns may be deactivated by applying a bias voltage of 0V and/or a voltage where the sensitivity of the imaging element is minimal. In this way, one or more columns and/or one or more rows may be deactivated.

This allows for the activation of single or multiple columns at once depending on the intended and/or evaluated imaging aperture, particularly with angular apertures. Notably, this is not possible with classical signal multiplexing circuits. Additionally, different bias voltages may be used to tune the imaging frequency higher or lower, or to combine adjacent rows and/or columns with differently tuned frequencies for harmonic tuning, which enables flexible acquisition schemes to improve imaging contrast and resolution.

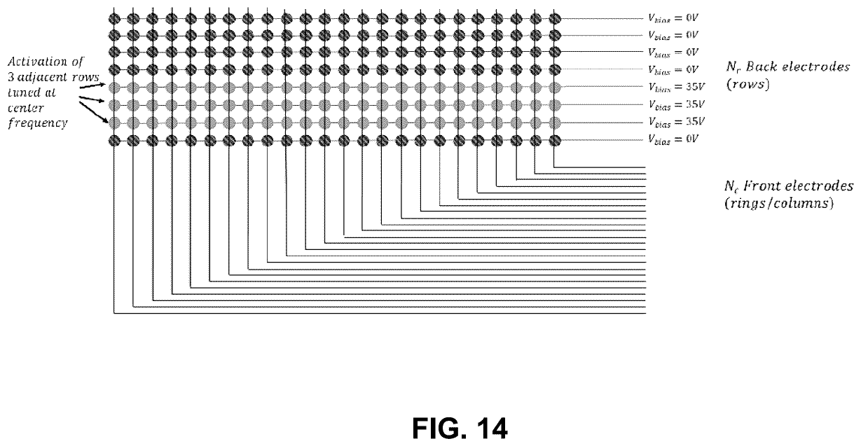

FIG. 14 illustrates an embodiment of activation of multiple rows of an imaging array of the invention. Notably, multiple adjacent rows, in this case three adjacent rows, may be tuned at a same center frequency. In this embodiment, back electrodes may activate Nr rows and front electrodes may activate Nc rings/columns. A bias voltage of 0V (and/or a voltage where the sensitivity of the imaging element is minimal) may be applied to rows not activated. This concept may be applied for ring/column activation as well. Importantly, the total number of interconnects is Nr+Nc, which results in a much smaller number of connections for arrays with high element counts in rows (Nr) and columns (Nc).

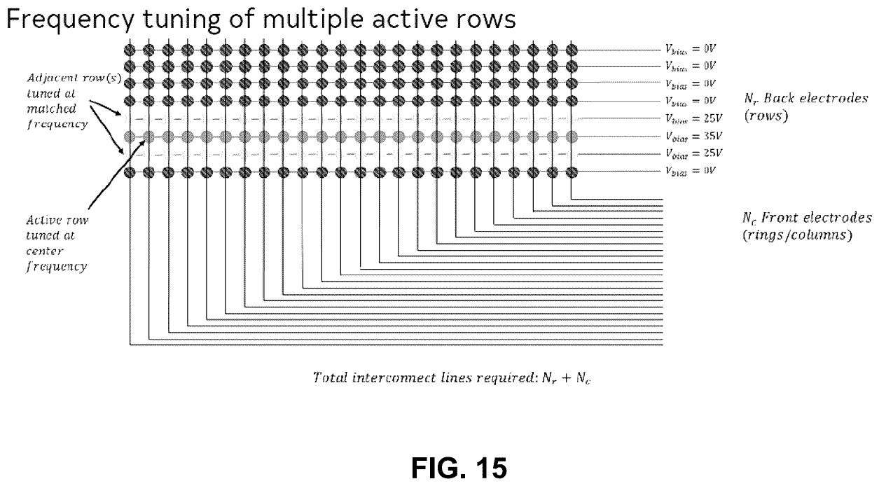

FIG. 15 illustrates an embodiment of frequency tuning of multiple active rows in an imaging array of the invention. In this example, back electrodes may activate Nr rows and front electrodes may activate Nc columns/rings. A selected row may be activated and tuned at a center frequency, while selected adjacent row(s) may be tuned at a matched frequency. Other selected rows may be deactivated by applying a bias voltage of 0V and/or a voltage where the sensitivity of the imaging element is minimal. As illustrated again, the total number of interconnects to achieve the desired frequency tuning for the angular aperture is Nr+Nc. Thus, in systems of the invention, the angular imaging aperture may be defined by the number of rows (Nr) and/or the number of columns (Nc) to which the bias voltage is applied such that a number of interconnections between imaging elements is Nr+Nc.

As noted above, the bias voltage level may be adjustable to tune a frequency of the imaging elements. Further, the bias voltage level selectively applied to the plurality of first and second electrodes may be the same or different for each electrode to allow for tuning the imaging frequency of the imaging elements higher and/or lower to achieve a desired frequency. Additionally, a positive bias voltage and/or a negative bias voltage level selectively applied to the plurality of first and/or second electrodes tunes an imaging operating mode.

Thus, using the principle described above, the use of row-column addressing of imaging elements in the array in combination with bias activation achieves a desired angular aperture for ultrafast imaging. Specifically, applying varying transmit/receive/biasing patterns subsequentially during imaging allows for targeted ultrafast imaging. As a result, the invention provides for defining and optimizing an angular aperture for ultrafast imaging that is not limited to a 90 by 60 degrees sector opening for a pyramidical volume view.

### Array Element Interfacing and System Architecture

The bias voltage selectivity of imaging systems and devices of the invention is controlled by the controller. The controller is capable of controlling a bias voltage selectively applied to one or more of the plurality of first and/or second electrodes to activate or deactivate imaging by the individual imaging elements in the row or column to define an angular imaging aperture.

The controller may include one or more interfaces, for example to operably connect the controller to aspects of the array and imaging electronics and to generate transmit pulse waves and convert the changes of the sensors into an equivalent quantity of voltage or current once wave data is received. The controller may also directly include these aspects of transmit and receive control (analog front-end). The controller may also be capable of controlling transmit and receive wave patterns for flexible image formation, or these capabilities may be integrated otherwise. Systems of the invention may include a plurality of interface modules such that each module is operably connected to both the controller and to one electrode in connection with a row or column.

In some embodiments, devices of the invention comprise a plurality of interface modules such that each module is operably connected to both the controller and to one electrode in connection with a row or column.

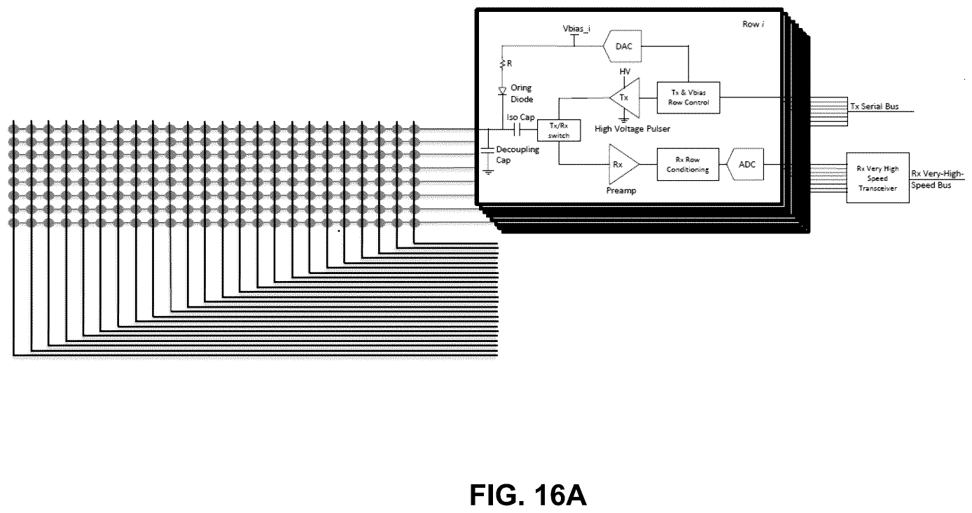

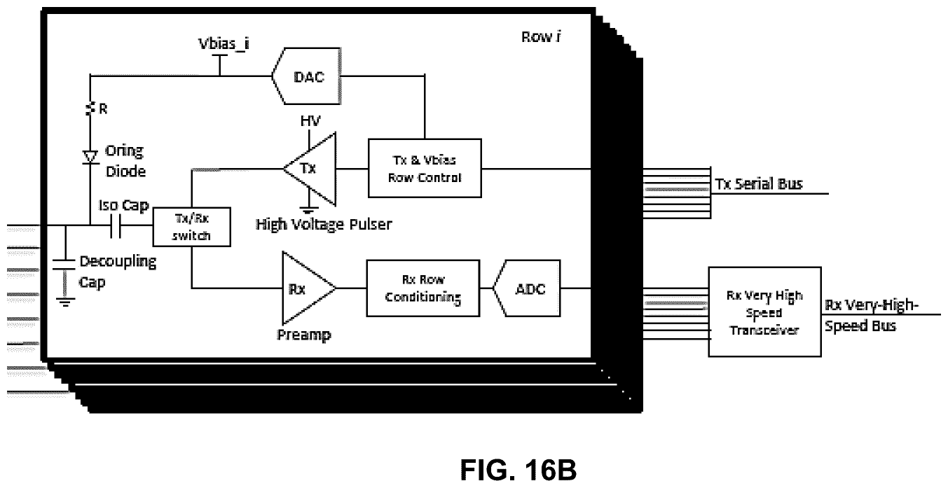

FIGS. 16A and 16B illustrate a diagram of one embodiment of the interface for a row of elements in an array. FIG. 16B shows an enlarged view of the circuit diagram of FIG. 16A. For example, the controller may include an interface for each electrode in connection with a row of individual imaging elements such that the bias voltage applied to the electrode is enabled, disabled, or defined by the interface. The controller may include an interface for each electrode in connection with a column of individual imaging elements such that the bias voltage applied to the electrode is enabled, disabled, or defined by the interface. These examples are to be understood as non-limiting in that systems of the invention may include a plurality of interface modules such that each interface module is operably connected to both the controller and to one electrode in connection with a row and/or column.

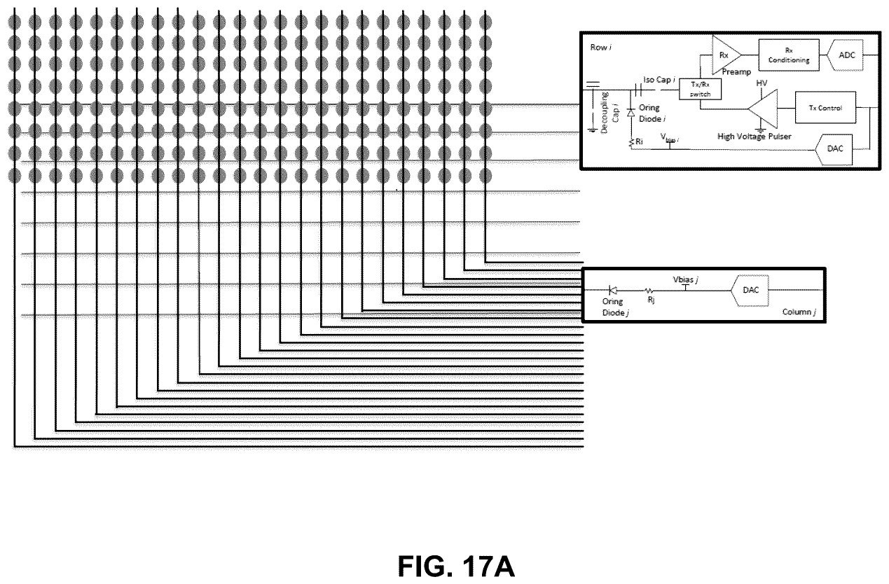

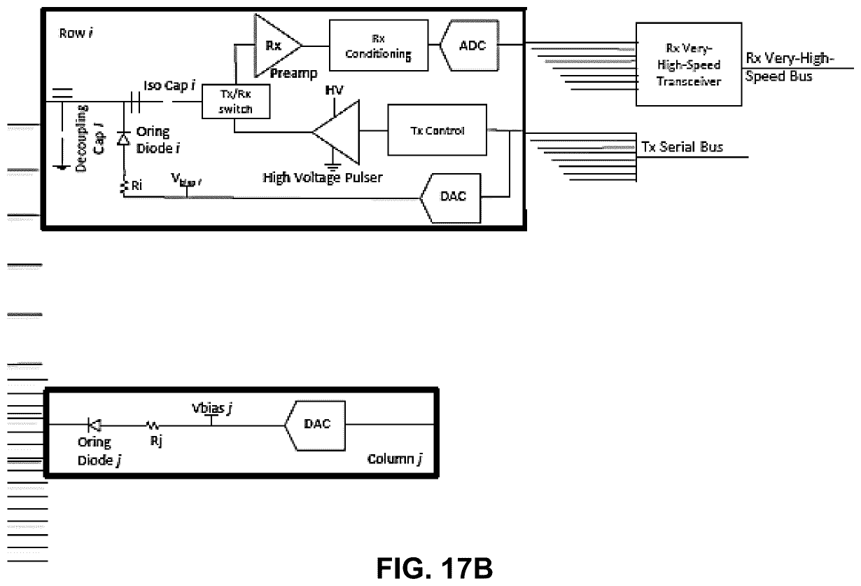

FIGS. 17A and 17B illustrate a diagram of one embodiment of imaging array element interfacing for a specific row and for a specific column, for example row i and column j, which results in a transmit or receive address for the element of, for example, (i, j). FIG. 17B shows an enlarged view of the circuit diagram of FIG. 17A. An interface may be included for row-column addressing for each row and/or column, and an interface may be included for bias activation of each column and/or row.

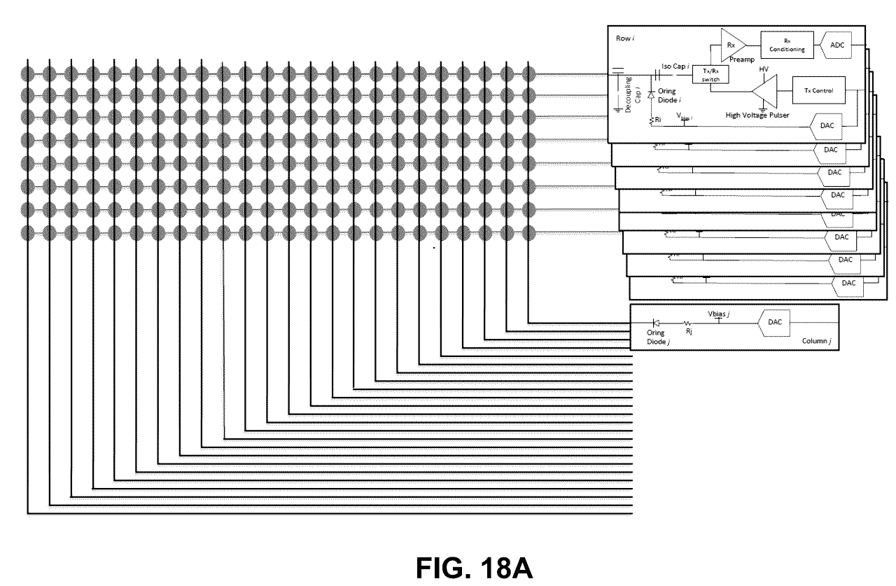

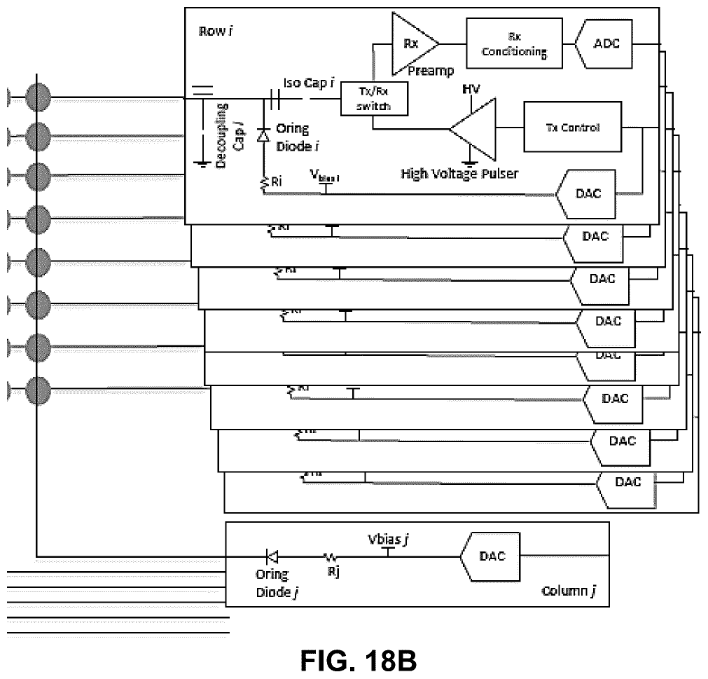

FIGS. 18A and 18B illustrate an embodiment of the invention in which each row of the imaging array is connected to an individual array element interface as is each column of the array such that individual imaging elements may be addressed for transmit and/or receive by a row and column (row, column) address. FIG. 18B shows an enlarged view of the circuit diagram of FIG. 18A.

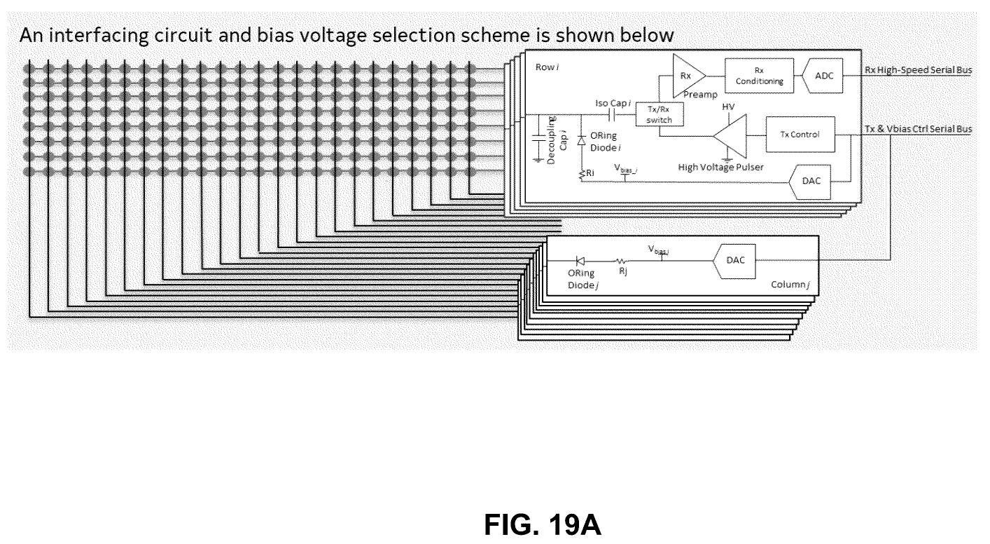

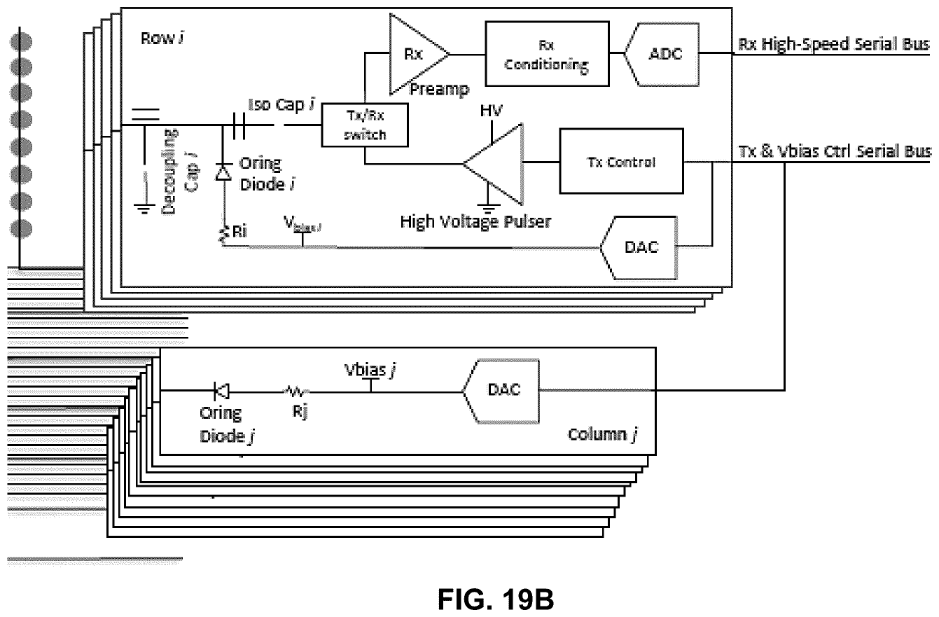

FIGS. 19A and 19B further illustrate an embodiment of the interfacing circuit with the bias voltage selection showing the interfacing circuit for the imaging element at row i, and column j, i.e. the imaging element at address (i, j). FIG. 19B shows an enlarged view of the circuit diagram of FIG. 19A. For bias voltage selection, each row and each column bias voltage may be individually enabled, disabled or set using a digital-to-analog converter (DAC) specific to each imaging element. The DACs may be addressable via a common transmit (Tx) and voltage bias (Vbias) control serial bus.

The controller may also include a protection circuit operably connected in series with each row and each column of individual imaging elements such that multiple bias voltage levels cannot be simultaneously applied to a given electrode. For example the protection circuit may be a fuse or circuit breaker or other circuit protection device. In some embodiments, the ORing circuits utilize diodes and/or transistors.

As an example, one or more ORing diodes may be placed in series with each DAC output to protect each element from simultaneous activation of bias voltage at its row address position and column address position. In this example, the ORing diodes are in series with a DAC to protect the element at address (i, j), from simultaneous activation of Vbias_i and Vbias_j. For non-CMUT elements, DACs may be disabled to achieve Vbias_i=Vbias_j=0V.

In some embodiments, one or more bias voltage selection circuits are connected to the controller using one or more multipoint communication interfaces. In some embodiments, the control of the transmit function and/or the receive function uses one or more interfaces that are common to or separate from the interfaces used for bias voltage selection.

The controller may include a computer program comprising an algorithm 201 for evaluating, calculating, and optimizing the angular imaging aperture. For example, using defined algorithms, the controller may be configured to provide feedback to an operator before or during operation of the imaging array for evaluating, calculating, and optimizing the angular imaging aperture. The controller may be configured to provide feedback after operation of the imaging array. The algorithm 201 may be part of a computer program executable by a computing system 203 and in communication with the controller of the imaging device.

The controller may be configured to activate single or multiple rows at once using the same or different bias voltage levels to achieve the optimized angular imaging aperture. The controller may be configured to activate single or multiple columns at once using the same or different bias voltage levels to achieve an intended angular imaging aperture.

The controller may be configured to control different activation and/or tuning schemes individually per transmit-receive event, where multiple transmit-receive events may have the same or alternating schemes, enabling flexible beamforming approaches.

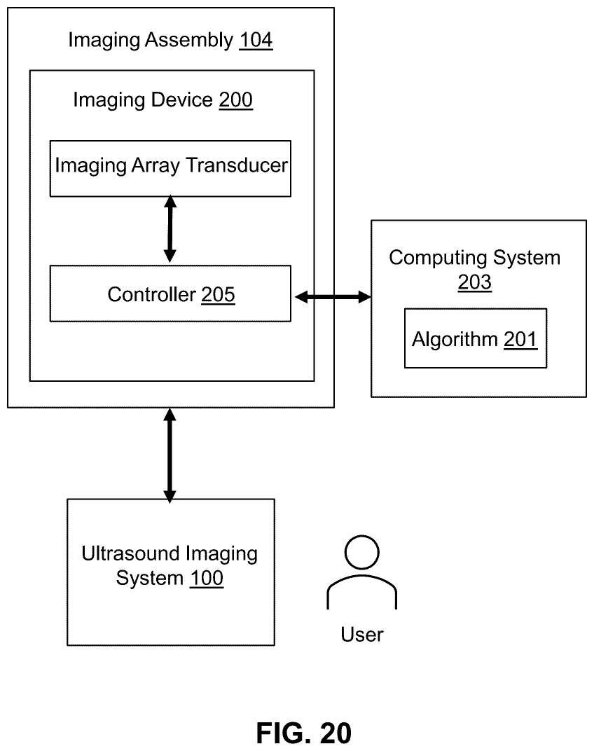

FIG. 20 illustrates an embodiment of an imaging device 200 of the invention in which the controller 205 is in active communication with a computing system 203 comprising an algorithm 201 for evaluating, calculating, and optimizing the angular imaging aperture. The device may be operably connected to an imaging assembly 104 in connection with an ultrasound system 100 as described in detail above. In this way, the controller, in active communication with a computing system, may be configured to activate single or multiple rows at once using the same or different bias voltage levels to achieve an optimized angular imaging aperture.

The controller may be configured to simultaneously activate multiple individual imaging elements based on the row address position and column address position of the respective individual imaging elements. Thus, the simultaneously activated individual imaging elements may have the same or different bias voltage levels.

To activate a desired row of imaging elements, the controller may select a transmit address corresponding to the desire row and send a signal with a desired bias voltage level to the interface for the desired row. Similarly, to activate a desired column of imaging elements, the controller may select a transmit address corresponding to the desired column and send a signal to the interface with a desired voltage level for the desired column.

For example, to activate all elements in a row, transmitting to only the desired row of elements may be achieved by selecting the transmit address of the Tx and Vbias Control bus. Activating all elements in row i, the transmit address corresponding to row i of the Tx & Vbias Ctrl bus may be selected. This enables and sets the value of row bias voltage. For row i, the bias voltage is designated as Vbias_i. Thus, the value of row I bias voltage (Vbias_i) may be enabled and set. Further, the column bias voltage of column j, designated as Vbias_j, may be disabled by setting Vbias_j equal to 0V for all columns. Notably, multiple rows may be activated simultaneously using the same or different bias voltage levels. In this way the imaging aperture for ultrafast imaging may be defined.

Similarly, to activate a single element at row i, column j, (i.e. (i, j)), the system allows for transmitting to all row elements by selecting the address corresponding to row i of the Tx & Vbias control bus. All other row bias voltage may be disabled by setting Vbias_i equal to 0V for all rows. The bias voltage of column j (Vbias_j) may then be enabled and set.

To activate all elements in column j, the system allows for transmitting to all elements by selecting all transmit (Tx) addresses of the Tx & Vbias Control bus, then disabling all row bias voltages by setting Vbias_i equal to 0V for all rows. Column j bias voltage (Vbias_j) may then enabled and set. In this way, multiple columns may be activated simultaneously using the same or different bias voltage levels.

The controller may also include an integrated circuit for bias voltage generation and control as well as other aspects of transmitting and receiving wave data (analog front-end circuit). The integrated circuit may be housed in an enclosure together with the imaging elements or positioned upon a substrate with the imaging elements. For example, the substrate may be a flexible substrate such as a polymer backing, compliant silicone elastomers, or other material capable of housing both the integrated circuitry as well as the imaging elements as is known to persons skilled in the art.

The controller may be housed separately from the imaging elements and may be operably coupled to the imaging elements via circuitry. For example the circuitry may include at least one of one or more cable assemblies, one or more printed circuits, and/or one or more flexible printed circuits.

Further, the controller may also include an analog front-end circuit, housed separately from the imaging elements, and including one or more signal generators and/or one or more signal transmitters, and/or one or more switching circuits packaged separately to the imaging elements or in a remote enclosure connected to the imaging elements via cable assemblies. For example, the analog front-end circuit (i.e. a set of analog conditioning circuitry using sensitive analog amplifiers) may use operational amplifiers, filters, or specific IC blocks for the sensors or other circuits to interface the sensors, analog-to-digital converters (ADC) or microcontrollers.

Systems and devices of the invention may have more than one controller. For example, besides the controller controlling bias voltage selectively, there may be a second (or the same) controller that transmits ultrasound pulses of a certain imaging frequency into tissue and then converts the received echoes into a digital signal. This may be understood as an ultrasound analogue frontend providing transmit and receive capabilities, which may be integrated with bias activation, or it may be implemented separately. Importantly, the transmit and receive functions of the ultrasound transmit and receive sequences may be controlled in synchrony with the controller for bias tuning and selection to allow for intelligent beamforming.

Besides the controller controlling bias voltage selectively, the controller may transmit ultrasound pulses of a certain imaging frequency into tissue and then convert the received echoes into a digital signal.

Systems and devices of the invention provide for the flexible arrangement of the controller and associated components to flexibly address the various size embodiments of device designs to suit particular applications. For example, the integrated circuit of the controller may be housed together with the analog front-end circuit at a catheter tip adjacent to the imaging elements. The integrated circuit may be housed together with the analog front end circuit operably coupled to the imaging elements and positioned directly adjacent to the imaging elements. In some embodiments, the signal generators, signal transmitters, and/or the switching circuits are housed in a remote enclosure connected to the imaging elements via circuitry. The circuitry may include, for example, cable assemblies, printed circuits, and/or one or more flexible printed circuits.

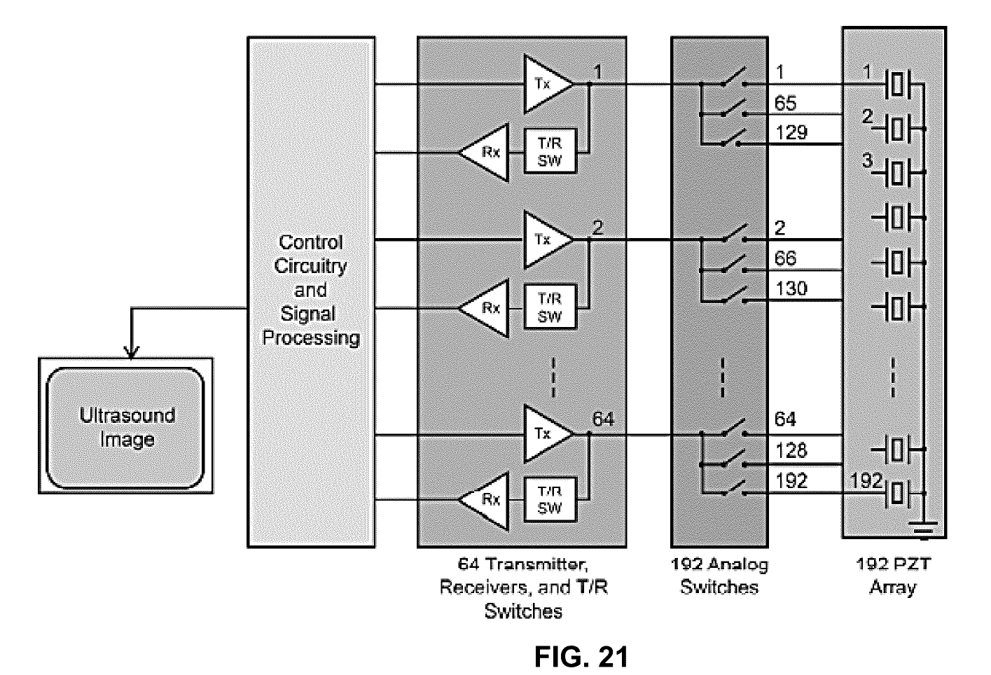

FIG. 21 diagrams another embodiment of systems of the invention to produce an ultrasound image using ultrafast imaging techniques.

As noted above, the plurality of imaging elements may be acoustic sensors selectively activated by the controller based on the row address and/or the column address of the acoustic sensor to transmit and/or receive a plurality of incident acoustic wave signals as wave data. The wave data may be, for example at least one of plane wave data and diverging wave data associated with one or more plane wave transmit-receive cycles carried out by the imaging elements. The active elements used for transmit and receive may comprise all elements in both transmit and receive, an identical subset of elements used for transit and receive, or a different subset (or full set) of elements used for transmit and receive of wave data.

The wave data may be full circumferential, three-dimensional (3D) image data. For example, using row-column addressing with bias activation, the array of acoustic sensors may be set at different transmission angular positions and thus transmit a plurality of incident acoustic wave signals representative of one or more plane waves of a plane wave group to aid in depiction of a 3D image over time.

The transducer of the system may be cylindrically-shaped, thus the array of individual imaging elements may be arranged as a plurality of rows longitudinally along the transducer and as a plurality of columns circumferentially around the transducer. As noted above, the imaging array may include a number of rows (Nr), a number of individual imaging elements per row (Ne), a row spacing, and a number of columns (Nc), such that the total number of imaging elements in the array is (Ne·Nr) and the individual imaging elements are connected through a number of connections represented by Nr+Nc. Nr connections may be used to transmit and receive ultrasound signals as ultrafast wave data and Nc connections may be used to control the bias voltage to the elements.

The cylindrical transducer may have thousands of elements allowing for ultrafast planewave or diverging wave imaging. For example, 64 columns (rings) and an angular spacing of 1 degree results 23,040 elements. Similarly, the cylindrical transducer may have 128 elements per row, and a row spacing of 1 degree yields 46,080 individual imaging elements. These elements may be connected through 488 connections (128+360), where 360 lines may be used to control the bias voltage, and 128 lines may be used to transmit and receive ultrasound signals as ultrafast wave data. Alternatively, both 360 lines and 128 lines (for rows and columns) may be used for both bias and transmit and receive voltage signals. This number of connections may be easily managed with wired connections, for example, in the catheter through a twisted pair, micro-coax of flexible printed circuit cable assembly. Thus, row (or column) activation allows for the flexible selection of groups of sub-elements (or rows of active elements) to define the angular imaging aperture.

The row spacing around the cylindrical array may be any plane angle. For example, the row spacing may be from about 0.1 degree to about 5 degrees in angular direction. The number of rows required for diverging wave may depend on the spacing of the rows, e.g., 5 degrees versus 0.1 degree, and the opening of the imaging wave. In practice, the row spacing may be determined as a balance of the angular spacing (0.1 to 1 degree in the example above) as well as the geometrical properties of a transducer element and the pattern of transducer elements. The size of a transducer element may have for example a size or diameter of 10 to 150 micro-meter, whereas the patten can be chosen accordingly to enable the said spacing of 0.1 to 1 degree as mentioned above.

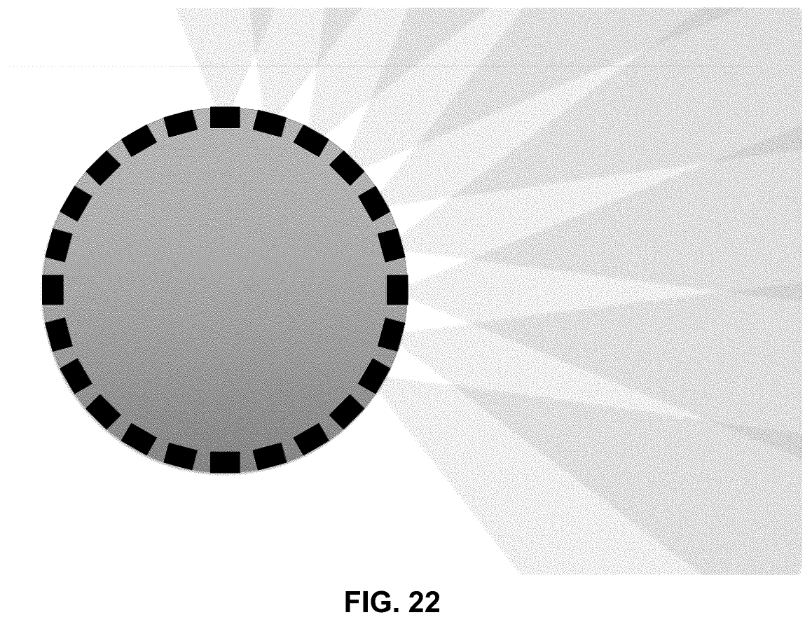

As illustrated in FIGS. 22 through 25, and noted above, an important differentiation of the invention as compared to other 2D array designs is that the invention is not required to transmit/receive on the full array. In fact, this would not provide any benefit for imaging resolution due to the radial aperture, that is, imaging of a certain side/view from the catheter may be sequential and not the full cylinder imaged at once.

FIG. 22 illustrates a cylindrical array transducer with 24 rows of elements radially around the transducer and the imaging aperture of nine elements that may be used for imaging a side of the transducer.

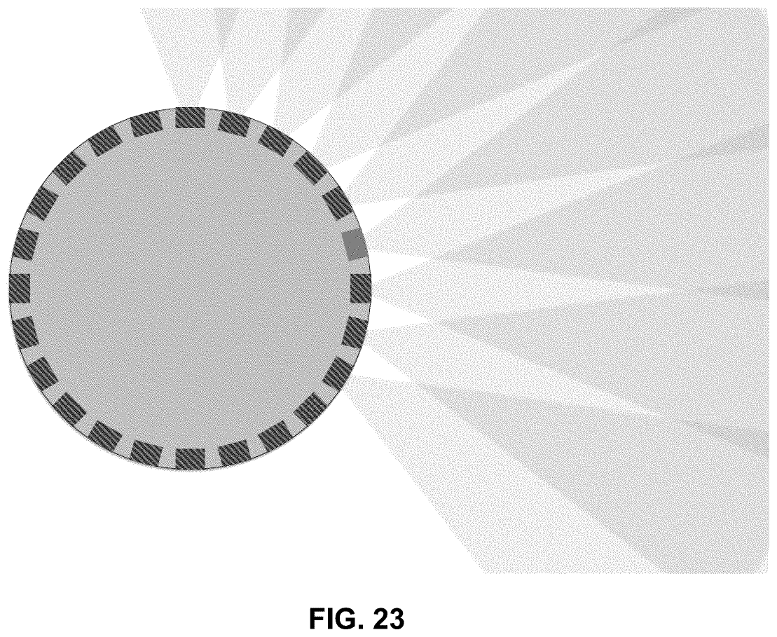

FIG. 23 illustrates the activation of a single element. Referring back to FIG. 4, the physical sensitivity aperture of an activated element is illustrated. Further, referring back to FIG. 5, the activation of three elements (or row of elements) to define an active convex aperture is illustrated.

As noted above, a bias voltage may be applied to a first electrode to activate a transmit and/or a receive function on the individual imaging elements connected to the second electrode such that the individual imaging elements transmit and/or receive ultrasound signals in the form of ultrafast wave data. In examples, the transducer array may include a number of individual imaging elements per row (Ne) in an array design allowing ultrafast plane wave and/or diverging wave imaging, such that the plane wave and/or diverging wave imaging mode comprises capturing reflected signal data at a rate of at least 10 kHz.

Generally, the transducer probe and/or the controller may be operably coupled to a console, which may generally control operation of the transducer probe (i.e., transmission of sound waves from the probe) and/or the controller. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

For example, in an exemplary embodiment, the console may generally include a computing device configured to communicate across a network. The computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for subsequent reconstruction into 2D and/or 3D images.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. The console may further provide control over an imaging assembly, including control over the emission of ultrasound pulses therefrom (intensity, frequency, duration, etc.) as well as control over the movement of the ultrasound transducer unit.

### Method for Optimizing an Imaging Aperture

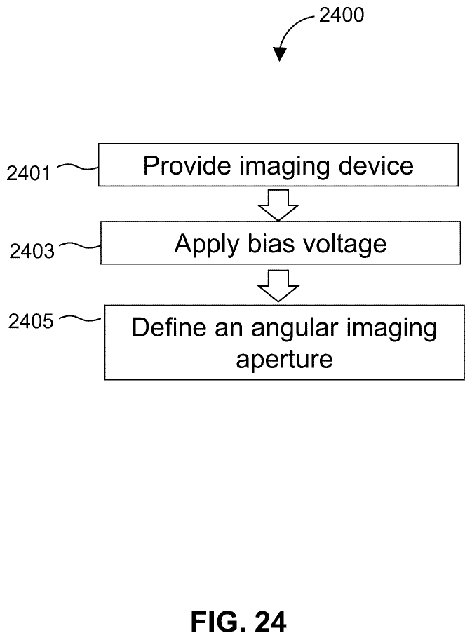

FIG. 24 illustrates an embodiment of a method for imaging 2400 of the invention. The method includes the steps of providing 2401 an imaging device as described in some embodiments herein, applying 2403 a bias voltage to selected electrodes to activate or deactivate selected imaging elements, and defining 2405 an angular imaging aperture.

Aspects of the invention provide a method for imaging which includes the steps of providing an imaging device as described herein comprising: a transducer comprising an array of individual imaging elements arranged as a plurality of rows longitudinally along the transducer and as a plurality of columns laterally along the transducer, wherein a signal connectivity of an individual imaging element is defined by a row address and a column address of the array; a plurality of first electrodes, wherein each first electrode is in connection with a row of individual imaging elements; a plurality of second electrodes arranged at a non-zero angle relative to the plurality of first electrodes, wherein each second electrode is in connection with a column of individual imaging elements; and a controller capable of controlling a bias voltage selectively applied to one or more of the plurality of first and/or second electrodes wherein the bias voltage defines a voltage for the row or column connected to the electrode to activate or deactivate imaging by the individual imaging elements in the row or column to define an angular imaging aperture. The method further comprises applying a bias voltage to selected electrodes to activate or deactivate selected imaging elements to define an angular imaging aperture.

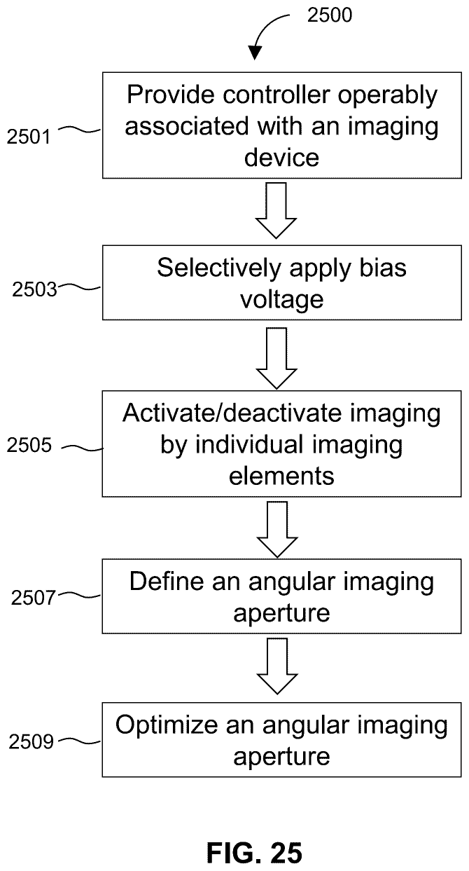

FIG. 25 illustrates a method for optimizing an angular imaging aperture 2500 of the invention.

Aspects of the invention also provide methods for optimizing an imaging aperture which include the steps of providing a controller operably associated with an imaging device 2501 as described herein comprising: a transducer comprising an array of individual imaging elements arranged as a plurality of rows longitudinally along the transducer and as a plurality of columns laterally along the transducer, wherein a signal connectivity of an individual imaging element is defined by a row address and a column address of the array; a plurality of first electrodes, wherein each first electrode is in connection with a row of individual imaging elements; a plurality of second electrodes arranged at a non-zero angle relative to the plurality of first electrodes, wherein each second electrode is in connection with a column of individual imaging elements. Further, the controller is coupled to non-transitory, computer-readable memory containing instructions executable by the processor to cause the controller to selectively apply a bias voltage level 2503 to one or more of the plurality of first and/or second electrodes. Thus, the methods include applying a bias voltage level to one or more selected electrodes such that the bias voltage level applied defines a voltage for the row or column connected to the electrode to activate or deactivate imaging 2405 by the individual imaging elements in the row or column to define 2507 an angular imaging aperture. The angular imaging aperture may then be optimized 2509.

As discussed above, systems and methods of the invention employ row-column addressing to tune an imaging aperture. Instead of connecting each element individually, all rows and all columns are connected together, allowing addressing of elements in a row-column fashion. For example, front electrodes on the imaging array may address columns (or “rings” on a cylindrical array) and back electrodes on the imaging array address “rows”. Notably, front and back electrodes of the imaging array may be switched for ring/row addressing respectively.

Additionally, the methods provide for using bias voltage to tune the frequency range of the imaging array with the sensitivity for a frequency range being driven by the bias voltage. Bias voltage can be used to a deactivate an imaging element entirely. Importantly, using bias voltage to activate imaging elements (i.e. cells) allows for imaging without multiplexing. Methods of the invention provide for using row-column addressing on the back (or front) electrodes to control the bias voltage applied to imaging elements to activate certain element groups dynamically, or to tune their frequency range sensitivity.

The bias voltage may be applied based on the row address and/or column address of one or more of the individual imaging elements. For example, a bias voltage may be applied to activate imaging in one or more rows. Likewise, a bias voltage may be applied to activate imaging in one or more columns.

Further, the method may include adjusting the bias voltage to tune a frequency of the imaging elements. The bias voltage selectively applied to the plurality of first and second electrodes may be the same or different for each electrode to allow for tuning the frequency of the imaging elements higher and/or lower to achieve a desired frequency.

A bias voltage of 0V may be applied for deactivation of a row, i.e. dampening element sensitivity to −60 dB. For example, applying a bias voltage of 0V or a voltage level where a sensitivity of the imaging elements is minimal may deactivate imaging in the row or column. For example, one or more columns and/or one or more rows may be deactivated, or alternatively all but one or more column and/or one or more rows may be deactivated, selectively activating an aperture of imaging elements.

To define an angular imaging aperture, methods of the invention allow for accessing the imaging elements as needed without carrying out fixed operations in sub-aperture groups. As noted above, using row-column addressing to define (i.e. tune) an angular imaging aperture of the array applies to any convex array surfaces with a certain radius. This concept may be applied to the special case of a convex surface fully around the imaging probe, i.e. a cylindrical surface. Therefore, the angular imaging aperture may be understood to mean the angular direction along a curvature.

In methods of the invention, angular imaging aperture may be defined as one or more rows or one or more columns of the imaging array based on a beam opening sensitivity of an individual imaging element of the array. Specifically the imaging aperture may be defined by the number of rows (Nr) and/or the number of columns (Nc) to which the bias voltage is applied such that a number of interconnections between individual imaging elements is Nr+Nc.

For example, the angular imaging aperture may be defined as one row or one column up to about 10 rows or 10 columns. The individual beam opening sensitivity may be known such that this value is included in calculations of a desired angular imaging aperture. For example, the individual beam opening sensitivity of an imaging element may be obtained through measurement and/or simulations based on the geometric and acoustic properties of the ultrasound imaging sensor array.

A bias voltage may be applied to front or back electrodes. For example, for Nr back electrodes, the electrodes may address rows for transmit and receive. Additionally, the bias voltage may be adjustable to tune the center frequency of the imaging elements. The bias voltage selectively applied to the plurality of first and second electrodes may be the same or different for each electrode to allow for tuning the imaging frequency of the imaging elements higher and/or lower to achieve a desired frequency.

For example, a bias voltage of 35V as a nominal voltage may be applied (tuned to 10 MHz imaging) applied to a row. Bias tuning (up or down) may be used to tune a center frequency. Nc front electrodes may address columns/rings for transmit and receive. A transmit pulse may be sent and data may be received for all rings/columns in a selected row. This allows for addressing elements within a row as single elements. A positive bias voltage and/or a negative bias voltage level selectively applied to the plurality of first and/or second electrodes may tune an imaging operating mode.

In some embodiments of the method, the imaging device may include a plurality of interface modules such that each interface module is operably connected to both the controller and to one electrode in connection with a row or column. Further, for bias voltage selection, each row and each column bias voltage may be individually enabled, disabled or set using a digital-to-analog converter (DAC) specific to each imaging element. The DACs may be addressable via a common transmit (Tx) and voltage bias (Vbias) control serial bus.

In some embodiments, one or more bias voltage selection circuits are connected to the controller using one or more multipoint communication interfaces. In some embodiments, the control of the transmit function and/or the receive function uses one or more interfaces that are common to or separate from the interfaces used for bias voltage selection.

The controller may also include a protection circuit operably connected in series with each row and each column of individual imaging elements such that multiple bias voltage levels cannot be simultaneously applied to a given electrode. For example the protection circuit may be a fuse or circuit breaker or other circuit protection device. In some embodiments, the ORing circuits utilize diodes and/or transistors.

One or more ORing diodes may be placed in series with each DAC output to protect each element from simultaneous activation of bias voltage at its row address position and column address position. In this example, the ORing diodes may be in series with a DAC to protect the element at address (i, j), from simultaneous activation of Vbias_i and Vbias_j. For non-CMUT elements, DACs may be disabled to achieve Vbias_i=Vbias_j=0V or Hz.

The controller may be configured to activate single or multiple rows at once using the same or different bias voltage levels to achieve an intended angular imaging aperture. In some embodiments, the controller may be configured to activate single or multiple columns at once using the same or different bias voltage levels to achieve an intended angular imaging aperture. The controller may be configured to simultaneously activate multiple individual imaging elements based on the row address position and column address position of the respective individual imaging elements. Thus, the simultaneously activated individual imaging elements may have the same or different bias voltage levels.

For example, in embodiments, the bias voltage selectively applied to one or more of the plurality of first and/or second electrodes enables or disables a transmit and/or receive function to define a transmit-receive event such that each transmit-receive event comprises an activation and/or a tuning scheme. Thus, the controller is configured to control the activation and/or tuning scheme individually for each transmit-receive event such that multiple transmit-receive events may have the same or alternating activation and/or tuning scheme.

The controller may include a computer program comprising an algorithm for evaluating, calculating, and optimizing the angular imaging aperture. For example, using defined algorithms, the controller may be configured to provide feedback to an operator before or during operation of the imaging array for evaluating, calculating, and optimizing the angular imaging aperture. The controller may be configured to provide feedback after operation of the imaging array. The algorithm 201 may be part of a computer program executable by a computing system 203 and in communication with the controller of the imaging device.

In this embodiment, the controller may be configured to activate single or multiple rows at once using the same or different bias voltage levels to achieve the optimized angular imaging aperture. The controller may be configured to activate single or multiple columns at once using the same or different bias voltage levels to achieve the optimized angular imaging aperture.

To activate a desired row of imaging elements, the controller may select a transmit address corresponding to the desire row and send a signal with a desired bias voltage level to the interface for the desired row. Similarly, to activate a desired column of imaging elements, the controller may select a transmit address corresponding to the desired column and send a signal to the interface with a desired voltage level for the desired column.

The bias voltage applied to a first electrode may activate the imaging elements connected to the second electrode for both a transmit function and a receive function. To activate all elements in a row, transmitting to only the desired row of elements may be achieved by selecting the transmit address of the Tx and Vbias Control bus. For example, activating all elements in row i, the transmit address corresponding to row i of the Tx & Vbias Ctrl bus may be selected. This enables and sets the value of row bias voltage. For row i, the bias voltage is designated as Vbias_i. Thus, the value of row I bias voltage (Vbias_i) may be enabled and set. Further, the column bias voltage of column j, designated as Vbias_j, may be disabled by setting Vbias_j equal to 0V or HZ for all columns. Notably, multiple rows may be activated simultaneously using the same or different bias voltage levels. In this way, the imaging aperture for ultrafast imaging may be defined.

The bias voltage applied to a first electrode may activate (i) a receive function of the individual imaging elements connected to the first electrode and a transmit function of the individual imaging elements connected to the second electrode; or (ii) a transmit function of the individual imaging elements connected to the first electrode and a receive function of the individual imaging elements connected to the second electrode.

The bias voltage applied to a second electrode may activate (i) a receive function of the individual imaging elements connected to the second electrode and a transmit function of the individual imaging elements connected to the first electrode; or (ii) a transmit function of the individual imaging elements connected to the second electrode and a receive function of the individual imaging elements connected to the first electrode.

The bias voltage applied to a first electrode may activate a transmit or receive function of the individual imaging elements connected to the first electrode and a transmit or receive function of the individual imaging elements connected to the second electrode.

A bias voltage applied to a first electrode may activate a transmit or receive function of the individual imaging elements connected to the first electrode and a transmit or receive function of the individual imaging elements connected to the second electrode.

The methods provide for the flexible arrangement of the controller and associated components to flexibly address the various size embodiments of device designs to suit particular applications. For example, the integrated circuit of the controller may be housed together with the analog front-end circuit at a catheter tip adjacent to the imaging elements. The integrated circuit may be housed together with the analog front end circuit operably coupled to the imaging elements and positioned directly adjacent to the imaging elements. The signal generators, signal transmitters, and/or the switching circuits may be housed in a remote enclosure connected to the imaging elements via circuitry. The circuitry may comprise cable assemblies, printed circuits, and/or one or more flexible printed circuits.

Devices of the methods may provide for a plurality of first electrodes in connection with a row of individual imaging elements, and a plurality of second electrodes arranged at a non-zero angle relative to the plurality of first electrodes.

The plurality of second electrodes may be arranged orthogonally relative to the plurality of first electrodes. For example, transmit and receive functions may be on the same electrode with activation of individual elements through bias (on/off) on the orthogonal electrode. This enables addressing of individual elements by bias voltage in combination with the orthogonal electrode. It should be noted that the transmit and receive functions may be on the same electrode or on orthogonal electrodes.

Additionally, the second set of electrodes may not be orthogonal to the first set of electrodes instead being any non-zero angle in relation to the first set of electrodes. Further, front and/or back electrodes may be switched in terms of interconnect (bias injection versus AC signal for transmit function).

The controller may include an integrated circuit housed within an enclosure together with the imaging elements or positioned upon a substrate together with the imaging elements. For example, the substrate may be a flexible substrate such as a polymer backing, compliant silicone elastomers, or other material capable of housing both the integrated circuitry as well as the imaging elements as is known to persons skilled in the art. In this embodiment, the controller may also include an analog front-end circuit, housed separately from the imaging elements, and including one or more signal generators and/or one or more signal transmitters, and/or one or more switching circuits. For example, the analog front-end circuit (i.e. a set of analog conditioning circuitry using sensitive analog amplifiers) may use operational amplifiers, filters, or ASICs for the sensors or other circuits to interface the sensors, analog-to-digital converters (ADC) or microcontrollers.

Alternatively, in some embodiments, the controller may be housed separately from the imaging elements and may be operably coupled to the imaging elements via circuitry. For example the circuitry may include at least one of one or more cable assemblies, one or more printed circuits, and/or one or more flexible printed circuits. Further, the controller may include an Analog Front End circuit comprising ultrasonic signal generators and transmitters, ultrasonic signal amplifiers and receivers, and switching circuits that may be packaged separately to the imaging elements or may be in a remote enclosure connected to the imaging elements via cable assemblies.

Devices of the method may have more than one controller. For example, besides the controller controlling bias voltage selectively, there may be a second (or the same) controller that transmits ultrasound pulses of a certain imaging frequency into tissue and then converts the received echoes into a digital signal. This may be understood as an ultrasound analogue frontend providing transmit and receive capabilities, which may be integrated with bias activation, or it may be implemented separately. The transmit and receive functions of the ultrasound transmit and receive sequences may be controlled in synchrony with the controller for bias tuning and selection to allow for intelligent beamforming.

The transducer of the method may be a micro-electromechanical systems (MEMS)-based capacitive micromachined ultrasonic transducer (CMUT) configured as a two-dimensional (2D) array structure. In non-limiting examples, the 2D array may be a flexible structure. Devices of the method may include a cylindrical imaging array which may consist of a flexible 2D-array structure in a CMUT design. The transducer may be made of an electrostrictive material configured as a two-dimensional (2D) array structure, or a different MEMS-based transducer configured as a two-dimensional (2D) array structure.

The transducer may be made of an electrostrictive material configured as a two-dimensional (2D) array structure.

The plurality of imaging elements may be acoustic sensors selectively activated by the controller based on the row address and/or the column address of the acoustic sensor to transmit and/or receive a plurality of incident acoustic wave signals as wave data. The wave data may be, for example, at least one of plane wave data and diverging wave data associated with one or more plane wave transmit-receive cycles carried out by the imaging elements. The active elements used for transmit and receive may comprise all elements in both transmit and receive, an identical subset of elements used for transit and receive, or a different subset (or full set) of elements used for transmit and receive of wave data.

The wave data may be full circumferential, three-dimensional (3D) image data. For example, using row-column addressing with bias activation, the array of acoustic sensors may be set at different transmission angular positions and thus transmit a plurality of incident acoustic wave signals representative of one or more plane waves of a plane wave group to aid in depiction of a 3D image over time.

For a cylindrically-shaped transducer, the array of individual imaging elements may be arranged as a plurality of rows longitudinally along the transducer and as a plurality of columns circumferentially around the transducer. The imaging array may include a number of rows (Nr), a number of individual imaging elements per row (Ne), a row spacing, and a number of columns (Nc), such that the total number of imaging elements in the array is (Ne·Nr) and the individual imaging elements are connected through a number of connections represented by Nr+Nc. Thus, in some embodiments, Nr connections are used to transmit and receive ultrasound signals as ultrafast wave data and Nc connections are used to control the bias voltage to the elements.

The cylindrical transducer may have thousands of elements allowing for ultrafast planewave imaging. For example, with 64 columns (rings) and an angular spacing of 1 degree, this would be 23,040 elements. Similarly, in other embodiments, the cylindrical transducer may have 128 elements per row, and a row spacing of 1 degrees which would yield 46,080 individual imaging elements. These elements may be connected through 488 connections (128+360), where 360 lines may be used to control the bias voltage, and 128 lines may be used to transmit and receive ultrasound signals as ultrafast wave data.

The row spacing around the cylindrical array may be any plane angle. For example, the row spacing may be from about 0.1 degree to about 5 degrees in angular direction.

The bias voltage may be applied to a first electrode to activate a transmit and/or a receive function on the individual imaging elements connected to the second electrode such that the individual imaging elements transmit and/or receive ultrasound signals in the form of ultrafast wave data. In specific embodiments, the transducer array may include a number of individual imaging elements per row (Ne) in an array design allowing ultrafast plane wave and/or diverging wave imaging, wherein the plane wave and/or diverging wave imaging mode captures reflected signal data at a rate of at least 10 kHz.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions, or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for optimizing an imaging aperture comprising:
a controller operably associated with an imaging device, wherein the imaging device comprises:
a transducer comprising an array of individual imaging elements arranged as a plurality of rows longitudinally along the transducer and as a plurality of columns laterally along the cylindrical transducer, wherein a signal connectivity of an individual imaging element is defined by a row address position and a column address position of the array;
a plurality of first electrodes each in connection with a row of individual imaging elements; and
a plurality of second electrodes arranged at a non-zero angle relative to the plurality of first electrodes each in connection with a column of individual imaging elements;
wherein the controller is coupled to non-transitory, computer-readable memory containing instructions executable by the processor to cause the controller to selectively apply a bias voltage level to one or more of the plurality of first and/or second electrodes, wherein the bias voltage level defines a voltage for the row or column connected to the electrode to activate or deactivate imaging by the individual imaging elements in the row or column to define an angular imaging aperture.

2. The system of claim 1, wherein the angular imaging aperture is defined by the number of rows (Nr) and/or the number of columns (Nc) to which the bias voltage is applied such that a number of interconnections between imaging elements is Nr+Nc.

3. The system of claim 1, further comprising a plurality of interface modules wherein each interface module is operably connected to both the controller and to one electrode in connection with a row or column.

4. The system of claim 3, wherein a bias voltage level for each row and/or each column is individually enabled, disabled, and set using the interface module for the row or column.

5. The system of claim 3, wherein to activate a desired row of imaging elements, the controller selects a transmit address corresponding to the desired row and sends a signal with a desired bias voltage level to the interface for the desired row.

6. The system of claim 3, wherein to activate a desired column of imaging elements, the controller selects a transmit address corresponding to the desired column and sends a signal to the interface with a desired voltage level for the desired column.

7. The system of claim 3, wherein the interface further comprises one or more ORing circuits in series with a digital to analog converter (DAC) output to protect each imaging element from simultaneous activation of bias voltage at its row address position and column address position.

8. The system of claim 7, wherein the ORing circuits utilise diodes and/or transistors.

9. The system of claim 1, wherein the controller further comprises a computer program comprising an algorithm for evaluating, calculating, and optimizing the angular imaging aperture.

10. The system of claim 9, wherein the controller is configured to activate single or multiple rows at once using the same or different bias voltage levels to achieve the optimized angular imaging aperture.

11. The system of claim 9, wherein the controller is configured to activate single or multiple columns at once using the same or different bias voltage levels to achieve the optimized angular imaging aperture.

12. The system of claim 1, wherein the controller is configured to activate single or multiple rows at once using the same or different bias voltage levels to achieve an intended angular imaging aperture.

13. The system of claim 1, wherein the controller is configured to activate single or multiple columns at once using the same or different bias voltage levels to achieve an intended angular imaging aperture.

14. The system of claim 1, wherein the controller simultaneously activates multiple individual imaging elements based on the row address position and column address position of the respective individual imaging elements.

15. The system of claim 14, wherein the simultaneously activated individual imaging elements have the same or different bias voltage levels.

16. The system of claim 1, wherein a bias voltage selectively applied to one or more of the plurality of first and/or second electrodes enables or disables a transmit and/or receive function to define a transmit-receive event wherein each transmit-receive event comprises an activation and/or a tuning scheme.

17. The system of claim 16, wherein the controller is configured to control the activation and/or tuning scheme individually for each transmit-receive event such that multiple transmit-receive events may have the same or alternating activation and/or tuning scheme.

18. The system of claim 1, wherein the controller comprises a protection circuit operably connected in series with each row and each column of individual imaging elements such that multiple bias voltage levels cannot be simultaneously applied to a given electrode.

19. The system of claim 1, wherein the controller comprises an integrated circuit housed within an enclosure together with the imaging elements or positioned upon a substrate together with the imaging elements.

20. The system of claim 1, wherein the controller is housed separately from the imaging elements and is operably coupled to the imaging elements via circuitry.

21. The system of claim 20, wherein the circuitry comprises at least one of one or more cable assemblies, one or more printed circuits, and/or one or more flexible printed circuits.

22. The system of claim 1, wherein the controller further comprises:
an integrated circuit for bias voltage generation and control, wherein the integrated circuit is housed in an enclosure with the imaging elements or positioned upon a substrate with the imaging elements; and
an analog front-end circuit, housed separately from the imaging elements, and comprising one or more signal generators, and/or one or more signal transmitters, and one or more switching circuits.

23. The system of claim 22, wherein the integrated circuit is housed together with the analog front-end circuit at a catheter tip adjacent to the imaging elements.

24. The system of claim 22, wherein the integrated circuit is housed together with the analog front end circuit operably coupled to the imaging elements and positioned directly adjacent to the imaging elements.

25. The system of claim 22, wherein at least one of the one or more switching circuits, the one or more signal transmitters, and the one or more switching circuits are housed in a remote enclosure connected to the imaging elements via circuitry comprising one or more cable assemblies, one or more printed circuits, and/or one or more flexible printed circuits.

26. The system of claim 1, wherein the bias voltage level is adjustable to tune a frequency of the imaging elements.

27. The system of claim 26, wherein the bias voltage level selectively applied to the plurality of first and second electrodes is the same or different for each electrode to allow for tuning the imaging frequency of the imaging elements higher and/or lower to achieve a desired frequency.

28. The system of claim 26, wherein a positive bias voltage and/or a negative bias voltage level selectively applied to the plurality of first and/or second electrodes tunes an imaging operating mode.

29. The system of claim 1, wherein applying a bias voltage of 0V or a voltage level where a sensitivity of the imaging elements is minimal deactivates imaging in the row or column.

30. The system of claim 29, wherein one or more columns and/or one or more rows are deactivated.

31. The system of claim 1, wherein the angular imaging aperture is one or more rows or columns defined based on a beam opening sensitivity of an individual imaging element of the array.

32. The system of claim 31, wherein the angular imaging aperture is defined as one row or column up to about 10 rows or columns.

33. The system of claim 1, wherein the transducer is a Micro-electromechanical systems (MEMS)-based capacitive micromachined ultrasonic transducer (CMUT) configured as a two-dimensional (2D) array structure.

34. The system of claim 33, wherein the 2D array structure is a flexible structure.

35. The system of claim 1, wherein the transducer comprises an electrostrictive material configured as a two-dimensional (2D) array structure.

36. The system of claim 1, wherein the plurality of imaging elements are acoustic sensors selectively activated by the controller based on the row address and/or the column address of the acoustic sensor to receive a plurality of incident acoustic wave signals as wave data.

37. The system of claim 36, wherein the wave data comprises at least one of plane wave data and/or diverging wave data associated with one or more plane wave transmit-receive cycles carried out by the imaging elements.

38. The system of claim 36, wherein the wave data is full circumferential, three-dimensional (3D) image data.

39. The system of claim 1, wherein the transducer is cylindrically shaped.

40. The system of claim 39, wherein the array of individual imaging elements is arranged as a plurality of rows longitudinally along the transducer and as a plurality of columns circumferentially around the transducer.

41. The system of claim 39, wherein the array comprises a number of rows (Nr), a number of individual imaging elements per row (Ne), a row spacing, and a number of columns (Nc), wherein the total number of imaging elements in the array is (Ne·Nr) and the individual imaging elements are connected through a number of connections represented by Nr+Nc.

42. The system of claim 39, wherein the row spacing is from about 0.1 degree to about 1 degree in angular direction.

43. The system of claim 39, wherein the bias voltage is applied to a first electrode to activate a transmit and/or a receive function on the individual imaging elements connected to the second electrode such that the individual imaging elements transmit and/or receive ultrasound signals in the form of ultrafast wave data.

44. The system of claim 43, wherein the transducer array comprises a number of individual imaging elements per row (Ne) in an array design allowing ultrafast plane wave and/or diverging wave imaging, wherein the plane wave and/or diverging wave imaging mode comprises capturing plane wave reflected signal data at a rate of at least 10 kHz.

45. The system of claim 1, wherein the plurality of second electrodes is arranged orthogonally relative to the plurality of first electrodes.

46. The system of claim 1, wherein one or more bias voltage selection circuits are connected to the controller using one or more multipoint communication interfaces.

47. The system of claim 1, wherein the control of the transmit function and/or the receive function uses one or more interfaces that are common to or separate from the interfaces used for bias voltage selection.
